(19) United States
(12) Patent Application Publication
(10) Pub. No.: US 2012/0097540 A1
(43) Pub. Date: Apr. 26, 2012

(54) MATERIALS AND METHODS FOR RESOLVING POLYHYDRIC SPECIES BY ELECTROPHORESIS

(76) Inventors: Tony David James, Bath (GB); John Fossey, Birmingham (GB); Johannes Maria Hubertus Van Den Elsen, Bath (GB)

(21) Appl. No.: 13/123,085

(22) PCT Filed: Oct. 9, 2009
(86) PCT No.: PCT/GB2009/002431

§ 371 Date: Nov. 8, 2011

(30) Foreign Application Priority Data
Oct. 10, 2008 (GB) 0818674.4

(51) Int. Cl. G01N 33/559

(52) U.S. Cl. 204/465; 204/456

(57) ABSTRACT

Materials and methods employing a polymerisable boronic acid species and a polymerisable linker are disclosed for making gels for resolving polyhydric species present in a sample by electrophoresis. The electrophoresis gels are capable of improving the effective separation of polyhydric species, especially those that show similar mobilities in standard electrophoresis or fluorophore-assisted carbohydrate electrophoresis (FACE). The use of template molecules in the reaction to form the electrophoresis gel with the boronic acid species and the polymerisable linker, so that the template molecule becomes incorporated into the electrophoresis gel, is also disclosed. The template molecule provides cavities in the electrophoresis gel that are generally complementary to the template molecule and which are adapted to reversibly interact with one or more of the polyhydric species present in the sample that have structures similar to the template molecule.

### FIELD OF THE INVENTION

The present invention relates to materials and methods for resolving polyhydric species by electrophoresis, and in particular to methods that employ gels that incorporate a boronic acid species.

### BACKGROUND OF THE INVENTION

Carbohydrates are life's most essential bioactive and information carrying molecules. As monomers or as part of larger glycoconjugates, carbohydrates have been shown to play vital roles in the biological processes of all organisms. In the last decades, carbohydrate analysis has become an ever more important challenge in medical biology, ranging from glucose monitoring and disease diagnosis (e.g. cancer and microbial infections) to their use as drugs and vaccines. Because of their characteristics, such as general electroneutrality (in most situations) and lack of chromophores or fluorophores, the detection and direct chemical analysis of carbohydrates are very difficult and more reliable analytical protocols are needed.

In post-translational modification of polypeptides, the polypeptides may be modified by the addition of carbohydrate components, for example by glycosylation or gluconoylation. Alternatively or additionally, they may be modified by phosphorylation. Unusual post-translational modification can be a marker for a disease or condition, for example cancer, and so reliable protocols for the analysis of the posttranslational modification of peptides also are needed.

Boronic acids have the general structure (I).

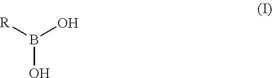

Boronate esters are made by simple by dehydration of boronic acid with alcohols. Boronate ester formation with diols is generally reversible, and this property offers the possibility of designing sensors and receptors for saccharides, which can be selective and sensitive for any chosen saccharide [1].

Usually, boronic acids interact with 1, 2 or 1,3 diols in the saccharide to form 5- or 6-membered cyclic boronic esters. Formation of this cyclic ester leads to an increase in the Lewis acidity of the boron atom, and this property enables the use of boronic acids as sensing or recognition molecules, for example by coupling to a fluorophore which changes its fluorescence in response to the change in Lewis acidity of the boron atom [2]. Scrafton et al [2] have proposed “click-fluors” employing a boronic acid conjugated to a 1,2,3-triazole ring, wherein binding of a saccharide to the boronic acid group switches on, or increases, fluorescence of the triazole donor.

D'Hooge et al [3] have described the synthesis of phenylboronic acid methacrylamides employing deprotecting a pinacolato boronic ester. The methacrylamide monomers can be used in the preparation of functional polymers for use in the carbohydrate recognition as discussed above.

Igloi and Koessel [4] have described the separation of RNA species using an affinity electrophoretic method using a covalently bound acryloylaminophenylboronic acid, present in concentrations of 2%, 5% and 10%.

With recent developments in the area of derivatisation of carbohydrates, methods for analysis of carbohydrates have made considerable progress [5]. This has led to the advance of a simple and sensitive method for the analysis of both mono- and oligosaccharides: fluorophore assisted carbohydrate electrophoresis (FACE) [6]. Whilst FACE is an excellent technique for analysis of different mass/charge sugars, the high-resolution separation of mixtures of saccharide molecules with a similar mass, structure and charge, as found in many biological samples, is still a challenge. Since the charged fluorescent labels that are necessary to separate carbohydrates on this basis affect the true nature of these complex species, neutral labels are more desirable. However, carbohydrates labelled with neutral fluorophores (such as 2-aminoacridone, AMAC) display unexpected migration properties in electrophoresis [7-9], and as such their usefulness is limited.

Accordingly, there remains a need for improved analytical protocols for carbohydrates and other polyhydric species and for assessing the post-translational modification of peptides, particularly employing neutral labels.

### SUMMARY OF THE INVENTION

Broadly, the present invention relates to materials and methods that employ a polymerisable boronic acid species that can be incorporated into gels for resolving one or more polyhydric species present in a sample by electrophoresis. The boronic acid species is generally incorporated into the gel by polymerising it with a polymerisable linker to produce a copolymer of the species. In particular, the present inventors found that the incorporation of boronic acid species, such as methacrylamido phenylboronic acid, in electrophoresis gels helped to improve the effective separation of polyhydric species, especially those that show similar mobilities in standard electrophoresis or fluorophore-assisted carbohydrate electrophoresis (FACE). Furthermore, gel electrophoresis using boronic acid species, even at low loading (typically 0.1% to 1.9% dry weight) altered retention of carbohydrate-containing species depending on their boronate affinity. By way of example, while conventional fluorophore-assisted carbohydrate electrophoresis of 2-aminoacridone labelled glucose oligomers shows an inverted parabolic migration, an undesired trait of small oligosaccharides labelled with this neutral fluorophore, boron affinity saccharide electrophoresis (referred to herein as “BASE”) separation according to the present invention completely restores the predicted running order of these carbohydrates, based on their charge/mass ratio, and results in improved separation of the analyte saccharides. Additionally, the present inventors have shown that gluconoylated, glycosylated and phosphorylated proteins can be separated by boron affinity electrophoresis.

In a further refinement, the present invention also includes the use of template molecules in the reaction to form the electrophoresis gel with the boronic acid species and the polymerisable linker, so that the template molecule becomes incorporated into the electrophoresis gel. Generally, where the template molecule is a polyhydric species, this will be via the formation of boronic esters with the boronic acid species in the gel as discussed further below. The template molecules can then be removed from the gel, for example by being displaced during an electrophoresis experiment, e.g. by buffer, or in separate washing step with a solvent. In either case, the template molecule provides cavities in the electrophoresis gel that are generally complementary to the template molecule and which are adapted to reversibly interact with one or more of the polyhydric species present in the sample that have structures similar to the template molecule. This generally has the advantage of improving the separation using the gel of such polyhydric species from those with structures that are dissimilar to the template molecule. Without wishing to be bound by any particular theory, the improved separation is believed to result from the polyhydric species in the sample having similar structures to the template molecule interacting with the cavities in the gel, and thereby being retarded compared to dissimilar polyhydric species.

Accordingly, in a first aspect, the present invention provides a method of resolving a polyhydric species present in a sample by gel electrophoresis, the method comprising:
(a) loading an electrophoresis gel with the sample containing the polyhydric species, wherein the electrophoresis gel is formed from a copolymer of a boronic acid species and a polymerisable linker;
(b) applying an electric field across the gel to cause the polyhydric species to migrate across the gel;
wherein the boronic acid species reversibly interacts with the hydroxyl groups present in the polyhydric species to cause different polyhydric species migrate through the gel at different speeds. Preferably, the boronic acid species is present in the copolymer between 0.1% and 1.5% dry weight.

Accordingly, as discussed further herein, the present invention may help to improve the resolution of different polyhydric species according to their charge/mass ratio and/or boronate affinity.

In some embodiments, the reaction to form the electrophoresis gel includes a template molecule that becomes incorporated into the electrophoresis gel to provide cavities in the gel that are adapted to reversibly interact with one or more of the polyhydric species present in the sample having a structure which is similar to the template molecule. In this case, the template molecule is preferably a polyhydric species that forms boronic esters or boronic ester analogues with the boronic acid species.

In some aspects, the methods of the present invention may be employed for detecting one or more of the polyhydric species separated on the gel. The detecting step can include detecting the presence or amount of one of more of the species on the gel. This may be done for a range of different purposes including detection of disease markers and diagnosis of disease. Additionally, the methods may be used to detect and/or separate glycated (non-enzymatically glycosylated; glycoxidated) peptides and proteins, for example in the food industry.

For example, the method may comprise correlating the presence or amount of one or more of the polyhydric species as a marker of a disease, condition or biological process, such as diabetes, cardiovascular disease, Alzheimer's disease, cancer, microbial infection and ageing, including diabetes-related aging.

Accordingly, in a further aspect, the present invention provides a method for diagnosing a patient suspected of having a disease associated with a polyhydric species, the method comprising:
(a) loading an electrophoresis gel with a sample containing the polyhydric species obtained from the patient, wherein the electrophoresis gel is formed from a copolymer of boronic acid species and an polymerisable linker;
(b) applying an electric field across the gel to cause the polyhydric species to migrate through the gel, wherein the boronic acid species reversibly interacts with hydroxyl groups present in the polyhydric species to cause different polyhydric species to migrate through the gel at different speeds, thereby allowing the polyhydric species to be resolved;
(c) detecting the polyhydric species resolved on the gel;
(d) correlating the presence or amount of one or more of the polyhydric species as a marker of a disease or condition. Again, it is preferable that the boronic acid species is present in the copolymer between 0.1% and 1.5% dry weight.

It is normal in gel electrophoresis for the polymer from which the gel is formed to be dissolved in a solvent by heating the mixture to produce a solution, typically in a microwave. Accordingly, in some embodiments, the methods described herein may include one or more initial steps carried out before the sample is loaded onto the gel. These step may comprise:
(i) mixing the copolymer of the boronic acid species and the polymerisable linker with a solvent for casting the gel; and/or
(ii) dissolving the copolymer in the solvent; and/or
(iii) casting the solution to produce the gel.

In a further aspect, the present invention provides a method of making a gel for resolving a polyhydric species present in a sample by gel electrophoresis, the method comprising:
(i) mixing a copolymer of boronic acid species and a polymerisable linker with a solvent for casting the gel; and/or
(ii) dissolving the copolymer in the solvent; and/or
(iii) casting the solution to produce the gel;
   wherein the boronic acid species is capable of reversibly interacting with hydroxyl groups present in the polyhydric species to cause different polyhydric species in the sample to migrate through the gel at different speeds.

The methods of the invention may further comprise the initial step of forming the copolymer from the boronic acid species, the polymerisable linker and optionally a polymerisable cross-linker.

In another aspect, the present invention provides electrophoresis gels for use in the resolving and diagnosis methods of the invention. Accordingly, the present invention provides an electrophoresis gel for resolving polyhydric species, the electrophoresis gel being obtainable by copolymerising a boronic acid species capable of polymerisation with a polymerisable linker.

In a further aspect, the present invention provides a kit for resolving polyhydric species, so suitable for use in the methods of this invention. The kit may comprise a polymerisable boronic acid species and a polymerisable linker for forming a copolymer for casting into an electrophoresis gel,
wherein during electrophoresis the boronic acid species reversibly interacts with the hydroxyl groups present in the polyhydric species to cause different polyhydric species migrate through the gel at different speeds.

Alternatively or additionally, the kit may comprise a dry copolymer of a boronic acid species and a polymerisable linker for casting into an electrophoresis gel, wherein during electrophoresis the boronic acid species reversibly interacts with the hydroxyl groups present in the polyhydric species to cause different polyhydric species migrate through the gel at different speeds.

Embodiments of the present invention will now be described in more detail by way of example and not limitation with reference to the accompanying figures.

### BRIEF DESCRIPTION OF THE FIGURES

FIG. 1. Rapid and reversible covalent saccharide binding with phenyl boronic acid.

FIG. 2. Compound 1 is acrylamide, an example polymerisable linker; Compound 2 is methylene bisacrylamide, an example cross-linker; Compound 3 is protected methacrylamide phenyl boronic acid; Compound 4 is phenyl methacrylamide.

FIG. 3. Synthesis MPBA (compound 3, FIG. 2) via compounds 5 (3-aminophenylboronic acid) and 6 (3-(5,5-dimethyl-1,3,2-dioxaborinin-2-yl)benzeneamine; for detailed description of the synthesis and analysis see Experimental section.

FIG. 4. Graph showing the speed of saccharide versus MPBA content in a 20% acrylamide gel

FIG. 5. Graph showing speed of saccharide versus Log of Mᵣ of saccharide in 20% acrylamide gel with MPBA concentrations ranging from 0-1%. The glucose oligomers analysed are, from left to right, glucose; maltose; maltotetraose; maltopentaose; maltohexaose and maltoheptaose.

FIG. 6. FACE separation profile of AMAC-labelled glucose oligomers. Lane 1: Glucose (G₁); lane 2: Maltose (G₂); lane 3: Maltotetrose (G₄); lane 4: Maltopentose (G₅); lane 5: Maltohexose (G₆); lane 6: Maltoheptose (G₇); lane 7: Glucose oligomer mixture. (A) Separation profile in the absence of boronate acrylamide in a 20% polyacrylamide gel. (B) Separation profile in the presence of 0.5% boron acrylamide incorporated in a 20% polyacrylamide gel. (C) Plot showing exponential retention increase of the glucose oligomers in the acrylamide gels versus boron-content (% of MPBA, w/w) (see also Supporting Information). (D) Separation profile in the presence of 0.5% N-phenylmethacrylamide incorporated in a 20% polyacrylamide gel.

FIG. 7. FACE separation profile of AMAC-labelled mono and disaccharides. Lane 1: Saccharide mixture; lane 2: Lactose; lane 3: Galactose; lane 4: N-acetyl glucosamine; lane 5: Melibiose; lane 6: Glucose. (A) Separation profile in the absence of boronate acrylamide in a 20% polyacrylamide gel, at pH 8.3. (B) Separation profile in the presence of 0.5% boron acrylamide incorporated in a 20% polyacrylamide gel, at pH 8.3. (C) Separation profile in the absence of boronate acrylamide in a 20% polyacrylamide gel, at pH 9.0. (D) Separation profile in the presence of 0.5% boron acrylamide incorporated in a 20% polyacrylamide gel, at pH 9.0.

FIG. 8. The SDS-PAGE analysis of glycosylated proteins at increasing MPBA concentrations. Lane 1: MW marker; Lane 2: C4c; Lane 3: B2GPI; Lane 4: Sbi-III-IV.

FIG. 9. 9A: Mass spectrometry analysis of recombinant Sbi construct Sbi-E showing an expected peak with a molecular weight of 30.7 kDa and a larger peak of 30.9 kDa, indicative for a δ-gluconolactone modification (molecular weight increase: 178 Da). 9B: SDS-PAGE analysis of recombinant protein Sbi-III-IV expressed in E. coli strain BL21 DE3, comparing gel profiles in the absence (left) and presence of methacrylamido phenylboronic acid MBPA (0.5%, right) of freshly produced and purified protein (lane 1) and Sbi-III-IV after 15′ (lane 2) and 16 h (lane 3) incubation with 100 mM δ-gluconolactone. After incubation with gluconolactone the intensity of the gluconylated Sbi product increases and the maximum intensity is reached after 15 seconds.

FIG. 10. Retention of gluconoylated Sbi-III-IV in SDS PAGE as a function of MPBA content. The position of the glyconoylated Sbi-III-IV band is expressed as virtual molecular weight (as compared with the molecular weight marker gel profile; left panel) and as relative molecular weight (as compared with the actual molecular weight of gluconoylated Sbi-III-IV; right panel).

FIG. 11. SDS-PAGE analysis, in the presence of 0.5% MPBA, of intact recombinant protein construct Sbi-III-IV and Sbi-III-IV with cleaved histidine tag after incubation with tobacco etch virus (TEV) protease. In the intact Sbi-III-IV construct (lane 1) the gluconoylated protein fraction appears to have a molecular weight of ˜60 kDa (indicated with black asterisk), in the cleaved protein (lane 2) the high molecular weight band of the δ-gluconolactone modified Sbi-III-IV has disappeared and a new low molecular weight appears at the expected position of the cleaved histidine-tag (white asterisk), which can be removed using nickel-ion chelating chromatography (lane 3).

FIG. 12. SDS-PAGE analysis in the absence and presence of MPBA showing the separation of the δ-gluconolactone modified E2 enzyme of a putative acetoin dehydrogenase complex from Sulfolobus solfataricus (SSE) from its non-glycated precursor, using gel according to the present invention. The SSE construct contains a 10-histidine affinity tag (with sequence: MGHHHHHHHHHHSSGHIDDD) and gluconoylation was confirmed by mass spectrometry (data now shown).

FIG. 13. 13A: SDS-PAGE analysis comparing the relative mobility of molecular marker (M) protein bands in a control and MPBA containing gel. Shown is the gel from FIG. 98 with “helper” lines to aid comparing the relative positions of the molecular weight markers in the control gel with the gel containing 0.5% MPBA. Both gels were electrophoresed in the same gel tank, at the same time. 13B: Enlarged view of the gel, indicating the increased retention of β-lactoglobulin in the presence of 0.5% MPBA, relative to adjacent molecular marker bands. 13C Mobility of the molecular marker protein band in a 1% MPBA. The lactosylated β-lactoglobulin species separated in the gel are indicated with black arrows.

FIG. 14. SDS-PAGE analysis comparing gel profiles in the absence (left) and presence of MBA (0.5%, right) of proteins with different post-translational modifications: β-casein (phosphorylated/not glycosylated, lanes 1-3); human hemoglobin (not phosphorylated/not glycosylated, lanes 4-6); chicken ovalbumin (phosphorylated/glycosylated, lanes 7-9) and Sbi-III-IV (not phosphorylated/gluconoylated, lanes 10-13). The proteins were loaded onto the gel in three adjacent lanes to aid accurate comparison of their relative mobilities.

FIG. 15. A schematic showing how molecular imprinting is employed in the present invention to create gels with structures adapted to bind specific polyhydric species.

FIG. 16. A SDS-PAGE experiment showing the improved separation of fructosamine-HSA from unglycated HSA on a gel templated using fructose.

### DETAILED DESCRIPTION

### Boronic Acid Species

The synthesis of boronic acid species suitable for use in accordance with the present invention is disclosed herein and other examples are available to the skilled person from the prior art. By way of example, a two-step deprotection of pinacolato methacrylamido phenylene boronic esters to generate 2-, 3- and 4-methacrylamido phenylboronic acids in good yield and purity is reported in [2].

The present inventors reasoned that inclusion of receptors that reversibly interact with polyhydric species, such as saccharides would advantageously affect retention characteristics, especially if the receptor displays differential interactions with diverse polyhydric species. The chosen receptor would need to be (i) easily incorporated into electrophoresis gels since covalent linking would prevent receptor leaching; (ii) able to differentially bind analyte polyhydric species such as saccharides and (iii) be tolerant to water.

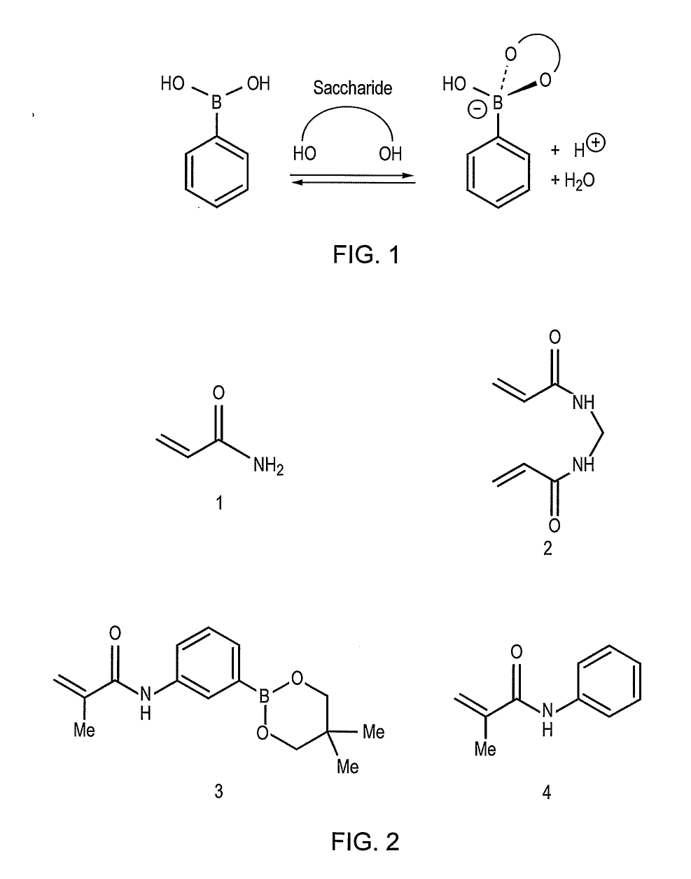

Boronic acids, particularly phenyl boronic acids, have the capacity to function as saccharide receptors in aqueous solution, attested by the many sensory systems reported [11-14]. They have been shown to form cyclic boronic esters with various polyhydric species such as carbohydrates under equilibrium conditions, via reversible covalent interactions in aqueous media, as is illustrated in FIG. 1. Boronic acids display differing binding affinities, independent of size/charge ratios and for this quality boronic acid-functionalised gels have been utilised as a stationary phase for the analysis and separation of monosaccharides, oligosaccharides and oligonucleosides by column chromatography [15]. Boronic acids have also been shown to interact with phosphate-containing species [16]

The boronic acid species may be a polymerisable boronic acid species, to allow it to copolymerise with a polymerisable linker to form a copolymer for forming an electrophoresis gel. The boronic acid species may be a boronic acid acrylamide, for example to facilitate the incorporation of the boronic acid species into an acrylamide electrophoresis gel.

A range of boronic acid species can be employed in the present invention. The boronic acids may be substituted or unsubstituted aryl boronic acids, such as substituted or unsubstituted phenyl boronic acids.

To prevent unwanted reaction of the boronic acid group during formation of the gels, the boronic acid species useful in the present invention include protected boronic acid species, such as boronate esters. For example, a suitable boronate ester is:

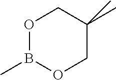

Preferred boronate esters include those formed by dehydration of boronic acid groups with alcohols. Preferably, the alcohols are diols which leads to the creation of cyclic boronate esters.

Particularly preferred boronic acid species are phenyl boron acid acrylamides and boronate esters thereof, which include ortho-, meta- and para-phenyl boronic acid acrylamides and esters thereof. Most preferred are ortho-, meta-, or para-methacrylamido phenylboronic acid and boronate esters thereof. Methacrylamido phenylboronic acid has the structure:

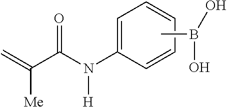

Meta-phenyl boronic acid acryalamides and boronate esters thereof may be preferred, for example meta-methacrylamido phenylboronic acid, which has the structure:

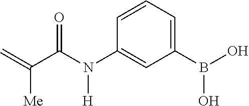

### Gel Electrophoresis

The use of gel electrophoresis for separating biomolecules such as proteins and nucleic acids is well known in the art and the techniques disclosed in reference textbooks such as Maniatis and Sambrook (Molecular cloning: a laboratory manual, 3rd edition, New York: Cold Spring Harbor Laboratory, 2001) and Ausubel et al. (Short Protocols in Molecular Biology, 5th Edition, A Compendium of Methods from Current Protocols in Molecular Biology. Wiley, 2002) may be adapted for use in accordance with the present invention. Other references cited herein describe the use of polyacrylamide gels for analysing saccharides [7-9]. In general, gel electrophoresis separates substances, most usually proteins, according to their electrophoretic mobility which is dependent on their size and length, molecular weight and other factors such as protein folding and post-translational modifications.

Electrophoresis gels are commonly a hydrogel assembled from a polymerisable linker such as acrylamide, and are often cross-linked by an agent, which may be a bisacrylamide monomer such as methylene bisacrylamide. These polymerization reactions may be adapted to produce the gels of the present invention by including the polymerisable boronic acid species to the electrophoresis gel preparation solution prior to polymerization.

In the gels of the present invention, it is preferable that the boronic acid species is present at low levels in the copolymer that forms the gel, typically at below 1.5% dry weight. By % dry weight, we mean the quantity of boronic acid species by dry weight present in the copolymer as a percentage of the monomers making up the copolymer. For example, when the copolymer is a copolymer of three types of monomers: boronic acid species, an acrylamide linker and a bisacrylamide cross-linker, typically 1.5% or less of the monomers by dry weight are boronic acid species.

The present inventors have found that at concentrations of more than about 1.0% dry weight, the gels run exponentially slower, and accordingly gels with high boronic acid species content undesirable. Therefore, it is preferable that the boronic acid species is present at 1.9% dry weight or less, more preferably at 1.1%, 1.2%, 1.3%, 1.4%, 1.5%, 1.6%, 1.7%, or 1.8% dry weight or less and most preferably at 1.0% dry weight or less. Preferably, the boronic acid species is present at 0.5% dry weight or more, more preferably at 0.4%, 0.3% or 0.2% dry weight or more, and most preferably at 0.1% dry weight or more. Similarly, when the copolymers are synthesised from their constituent monomers, it is preferable that they are used at the levels described above.

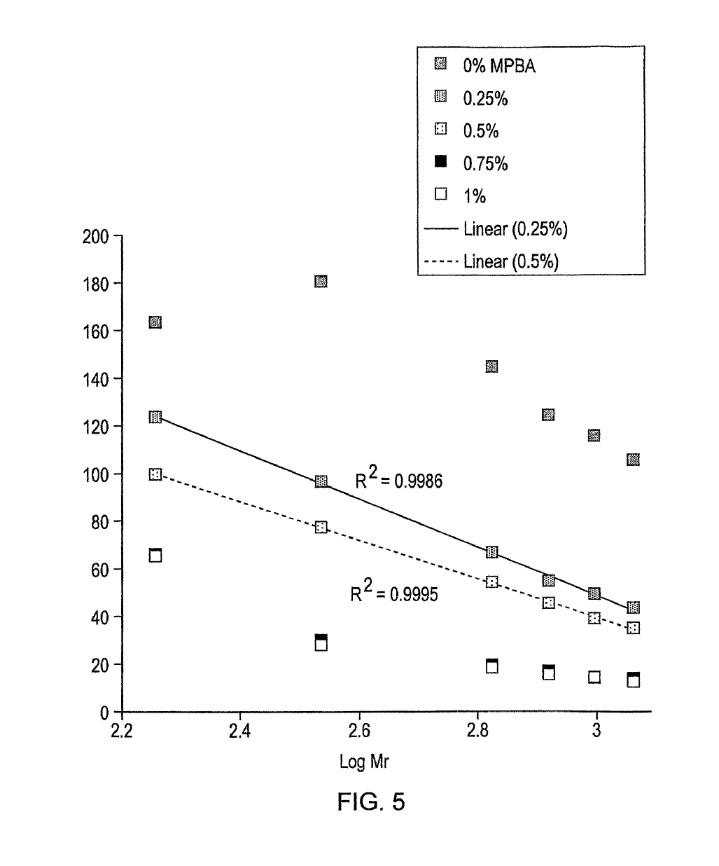

In addition, the present inventors have found that as the boronic acid species content of the copolymer forming the gel increases, the linerarity of the separation of polyhydric species may break down. For example, FIGS. 4 and 5 demonstrate this for the separation of saccharides on gels with varying methacrylate phenylboronic acid content, as discussed in Example 3.

Typically, the polymerisable boronic acid species may be added to the preparation solution at between 0.1% and 1.5% dry weight, and more preferably between 0.5%-1.0% dry weight. This ensures that the gel is formed from the polymerization of a polymerisable linker and a boronic acid species, i.e. so that the boronic acid species becomes covalently incorporated into the gel. This avoids leaching of the boronic acid species out of the gel.

A particularly preferred type of gel are those where the polymerisable linker is an acrylamide. Typically acrylamide linkers are used in combination with a polymerisable cross-linker such as a bisacrylamide monomer, for example methylene bisacrylamide, to produce electrophoresis gels. An initiator such as ammonium persulphate or TEMED is normally included to help to catalyse the polymerization reaction.

Alternatively or additionally, it is possible to employ molecular imprinting (MIP) techniques when making the electrophoresis gels of the present invention. MIP techniques add a template molecule during the reaction to produce the electrophoresis gel, e.g. so that the template molecule is present in the reaction mixture when the copolymerization reaction between the boronic acid species and polymerisable linker takes place, with the result that the electrophoresis gel thus produced includes the template molecule within the geometric structure of the gel. The template molecule can then be removed from the gel, for example by being displaced in the course of an electrophoresis experiment using the gel or in separate washing step, e.g. with a solvent. The cavities in the gel provided by the template molecule are generally complementary to the size and/or shape of the template molecule. The use of this approach is reviewed in Bergmann & Peppas (Progress in Polymer Science 33, 271-288, 2008). An advantage of using MIP techniques is that it creates electrophoresis gels with geometric structures that are adapted to bind substrates or analytes that are added to the gel and have the same or similar structures to the template molecule. This improves the ability of the gel to separate species that are capable of interacting with the cavities left by the removal of the template molecule, from other species that contain different polyhydric species or are unglycated.

Typically, the template molecule is a polyhydric species as described herein, such as a saccharide. The template molecule is generally included during the copolymerisation reaction between the polymerisable linker and the boronic acid species, so that the hydroxyl groups present on the polyhydric species become bonded to the boronic acid species in the electrophoresis gel. The template molecule may then displaced from the gel in the course of an electrophoresis experiment, or be washed out in a separate step by buffer or a solvent. In a preferred embodiment, the template molecule becomes covalently bonded to the gel during the polymerisation reaction in the same way that polyhydric species interact with the boronic acid gel when it is employed to resolve them.

The template molecule may be chosen according to a number of different criteria that are dependent on the polyhydric species that are intended to be resolved or detected using the electrophoresis gel. By way of example, where a polyhydric species is known to comprise a particular saccharide group, this may be used as the template molecule. In a variation of this approach, a template molecule might be chosen that is similar, but not identical, to the saccharide group of the polyhydric species, and which is easier to make or obtain. This is demonstrated in Example 7, where fructose is used as a template molecule to produce an electrophoresis gel suitable for separating fructosamine-HSA from unglycated HSA.

In use, gels may be used in reducing or non-reducing formats characterized by the inclusion (or not) of an agent such as sodium dodecyl sulphate (SDS) for denaturing proteins. These formats may also be used in the methods of the present invention. SDS is a long chain detergent that interacts with proteins and applies a negative charge that is in proportion to molecular weight, minimising the contribution made by the structure of proteins to their electrophoretic mobility so that migration is a function of molecular weight.

### Polyhydric Species

As discussed above, the present invention relates to resolving polyhydric species. The polyhydric species which may be resolved by the methods of the present invention include those having a plurality of hydroxyl groups.

The polyhydric species can interact with the boronic acid species to reversibly form boronate esters or boronate ester analogues. Where the boronic acid species included in the gel is a boronate ester, the polyhydric species may interact may interact with the boronic acid species by displacing the group forming the initial boronate ester. The boronate esters or boronate ester analogues formed by interaction of the polyhydric species with the boronic acid species may be cyclic. Boronate ester analogues include species wherein one or both of the O atoms of the boronate group are attached to an atom which is not C. By way of example, boronate ester analogues include boronate phosphoesters, which may be formed by the interaction between a boronic acid species, and one or more hydroxyl groups of a terminal phosphate.

It is preferable that the polyhydric species contains two hydroxyl groups which are sufficiently close to interact with a boronic acid species as discussed above. In particular, it may be desirable that the polyhydric species comprises two hydroxyl groups in a 1, 1 or 1, 2 or 1, 3 or 1,4 positional relationship with each other. Hydroxyl groups in a 1,1 relationship are covalently attached to the same atom in the polyhydric species and those in a 1,2 relationship are covalently attached to adjacent atoms in the polyhydric species (i.e. atoms joined by one covalent bond). Similarly, hydroxyl groups in a 1,3 relationship are attached to atoms in the polyhydric species which are separated by a further atom, and hydroxyl groups in a 1,4 relationship are attached to atoms in the polyhydric species which are separated by a further two atoms.

To facilitate the interaction of the two hydroxyl groups with the boronic acid species, it may be preferable that the hydroxyl groups are cis to each other. Hydroxyl groups in a cis relationship with each other include those which are positioned on the same side of a reference plane in the polyhydric species. For example, they could be located on the same face of a ring which forms part of the polyhydric species.

In some embodiments of the invention, the polyhydric species is a carbohydrate containing species. Carbohydrate containing species include species having moieties which contain carbon, oxygen and hydrogen atoms, such as saccharide moieties. For example, the species may contain moieties having the general formula Cₓ(H₂O)_{y}. Also included are moieties which are the deoxy forms of moieties having the general formula Cₓ(H₂O)_{y}, such as 2-deoxy-D-ribose, or oxidised forms of moieties having the general formula Cₓ(H₂O)_{y}, such as gluconolactone.

Carbohydrates are components of nucleosides, nucleotides, RNA and DNA, glycoproteins, glycolipids and glycosaminoglycans, and accordingly carbohydrate containing species include these species.

Carbohydrate containing species also include monosaccharides, oligosaccharides and polysaccharides.

In some preferred embodiments, the polyhydric species is selected from posttranslationally modified peptides, polypeptides and proteins, and mono-, oligo- and poly-saccharides.

In some embodiments, the polyhydric species is a phosphate containing species. Phosphate containing species includes species having the moiety —O—P(O)(OH)₂ irrespective of its state of ionisation.

The polyhydric species may be the product of posttranslational modification of polypeptides, as many types of such modification include hydroxyl groups that are capable of interaction with boronic acid species present in the gels disclosed herein. Posttranslational modification of polypeptides and proteins is discussed in more detail below.

The methods of the present invention may also be useful in identifying proteins that bind sugar molecules. Proteins incubated with sugar will be retained in the gel when the bound sugars interact with the boronic acid, provided that a non-denaturing gel is used. Accordingly, polyhydric species include proteins bound to sugar molecules by covalent, ionic and other non-covalent interactions such as hydrogen bonding.

As discussed above, different polyhydric species may migrate through the gel at different speeds in the methods of the invention. They may migrate through the gel at different speeds according to their mass/charge ratio and/or their boron affinity.

### Posttranslational Modification

The present invention may also be used for the detection of post-translational modification of peptides, polypeptides and proteins. Many types of posttranslational modification involve the covalent attachment of moieties comprising hydroxyl groups that are capable of interaction with boronic acid species present in the gels disclosed herein.

Posttranslational modification includes chemical modification of amino acids and the attachment of biochemical functional groups after their incorporation into polypeptides, during protein synthesis. This can, for example, have the effect of extending the range of function of proteins. Posttranslational modifications can control a protein's localization, turnover and active state structural changes and also manipulate their three-dimensional structure and interactions with other proteins. The analysis of these modifications is key to understanding the structure and function of proteins and protein-protein interactions. Accordingly, methods which allow the detection, characterisation and monitoring of posttranslational modifications will be of clear benefit to the study of protein structure and behaviour.

Undesired posttranslational modifications also may occur, for example, in the form of oxidation and glycation, the non-enzymatic attachment of sugars to proteins. Glycation is known as a biomarker for ageing and disease states related to diabetic complications [17-19]. The oxidised glucose derivative δ-gluconolactone, for instance, has been shown to cause glycation of hemoglobin, which may be a factor in the vascular complications of diabetes [20, 21]. The accumulation of δ-gluconolactone could play also play in important role in ageing processes [22] (see also [23]). Accordingly, methods which allow the monitoring and detection of posttranslational modifications may be useful in monitoring and/or diagnosis of diseases, conditions or biological processes.

Posttranslational modification also occurs in peptides, polypeptides and proteins expressed recombinantly. The posttranslational modification of recombinantly produced peptides, polypeptides and proteins may be different from the posttranslational modification of the same peptides, polypeptides and proteins when produced in native conditions (i.e. when produced by the organism which naturally produces the peptide). It is therefore highly desirable to be able to monitor and control posttranslational modification of recombinantly expressed peptides, polypeptides and proteins. Accordingly, methods which allow the detection, characterisation and monitoring of posttranslational modification of peptides, polypeptides and proteins will be of clear benefit to technologies involving recombinant expression, as will methods for the resolving and separating posttranslationally modified peptides, polypeptides and proteins. For example, control of post-translational gluconoylation in recombinant proteins is significant in the production of proteins of pharmaceutical and medical applications [24].

As discussed above, in many cases posttranslational modification of polypeptides and proteins may involve the introduction of moieties comprising a plurality of hydroxyl groups. Accordingly, polyhydric species include posttranslationally modified peptides, polypeptides and proteins, wherein the posttranslational modification may involve the introduction of a moiety comprising a plurality of hydroxyl groups. Introduction of a moiety by posttranslational modification includes covalent attachment of the moiety to the peptide, polypeptide or protein being modified.

The post-translationally modified peptide, polypeptide or protein may have been modified by the addition of carbohydrate components, for example by glycation, glycosylation or gluconoylation. Alternatively or additionally, the peptide, polypeptide or protein may have been modified by phosphorylation.

Accordingly, the polyhydric species of the present invention include glycated polypeptides and proteins, gluconoylated polypeptides and proteins, lactosyl polypeptides and proteins, phosphorylated polypeptides and proteins and glycosylated polypeptides and proteins.

The examples below show that the methods disclosed herein can be used to resolve, separate and detect glycation products such as δ-gluconolactone, as well as glycosylated and phosphorylated proteins.

Specific examples of posttranslational modification include, for example, spontaneous α-N-6-Phosphogluconoylation. This has been observed and described in recombinantly expressed proteins fused to a histidine affinity tag [25-27]. 6-phosphategluconlactone (6PGL) is an intermediate of the pentose phosphate pathway, which is produced by glucose-6-phosphate dehydrogenase (G6PD), and is a potent electrophile which reacts with the N-terminal amino group of histidine-tagged protein forming amine-linked product with the protein [27]. This modification has been shown to adversely affect protein activity [28] and interferes with crystallization of proteins [29]. It may also impair structure or immunogenicity of the expressed protein, which would greatly obstruct the use of recombinantly produced histidine-tagged proteins in research, diagnostics and therapy. As a model for analysing this modification, a protein construct based on Staphylococcus aureus immune-subversion protein Sbi may be used. This protein has been shown to inhibit the innate immune system [30] and is currently being developed as a therapeutic for complement-mediated acute inflammatory diseases. The Sbi-III-IV construct has a 25-residue N-terminal tag with sequence MSYHHHHHHDYDIPTTENLYFQGAM and mass spectrometry analysis of similar constructs containing this tag have shown that this sequence is specifically prone to 6-phosphogluconoylation. In the past, this undesired N-terminal adduct could only be detected by mass spectrometric analysis of the protein. The methods of the present invention may provide improved methods of detecting and separating peptides, polypeptides and proteins which have been subject to spontaneous α-N-6-Phosphogluconoylation.

Another example of posttranslational modification which introduces a moiety comprising a plurality of hydroxyl groups is formation of advanced glycation end products (AGEs), which starts with non-enzymatic addition of a sugar or a sugar-fragmentation product to a protein, followed by rearrangement to a linear Schiff-base adduct, finally rearranging to a protein-bound Amadori product. In later stages of the glycation process AGEs are formed, which may include a broad range of heterogeneous fluorescent and yellow-brown products, including nitrogen-containing and oxygen-containing heterocycles, resulting from subsequent oxidation and dehydration reactions [31,32]. It will be understood that the methods of the present invention may be used to resolve, separate monitor or detect one or more of the stages of the formation of AGEs described above, as each stage may involve the introduction or modification of moieties containing a plurality of hydroxyl groups.

AGEs are implicated in certain diseases and conditions, and may be markers of these diseases or conditions. Additionally, AGES may prove to be markers or indicators useful in monitoring biological processes such as ageing. As an example, β-amyloid deposits, the hallmarks of Alzheimer's disease, contain sugar-derived AGEs. Accordingly, the methods of the present invention may be useful in monitoring and detecting AGEs as markers associated with diseases, conditions and biological processes, or in monitoring and diagnosing diseases or conditions associated with AGEs. The methods may also prove useful in designing new inhibitors and/or drugs which can control, reduce or prevent the formation of AGEs, for example inhibitors of β-amyloid formation and drugs for treating Alzheimer's disease.

New methods for the analysis of posttranslational modifications may lead to better understanding of the process of posttranslational modification, which understanding may prove valuable in medical applications. For example, it has been found that β-amyloid deposits contain copper ions in addition to sugar-derived AGEs. It has also been shown in vitro that the formation of covalently cross-linked high-molecular-mass β-amyloid peptide oligomers, using synthetic β-amyloid peptide and glucose or fructose, is accelerated by micromolar amounts of copper (and iron) ions [33]. This finding may explain the specific formation of δ-gluconolactone adducts to N-terminal histidine metal-affinity tags in recombinant proteins, suggesting that histidine tag-bound metal ions could be involved in the acceleration of this process as well.

### Labels

Any label may be used to detect the polyhydric species resolved in methods according to the invention, and may be included in the kits of the invention. Neural labels are more desirable, because charged labels can affect the true nature of the polyhydric species. The label may be a visible or fluorescent label, to enable detection or visualisation of the polyhydric species resolved by the methods of the invention. In some embodiments, 2-aminoacridone (AMAC) is preferred. Alternative labels include 2-AA (2-aminobenzoic acid), 2-AB (2-aminobenzamide), DMB (diamino-4,5-methyleneoxybenzene), ANTS (8-aminonaphthalene-1,3,6-trisulfonic acid), and ANSA (1-amino-4-naphthalene sulphonic acid).

### Applications

The materials and methods disclosed herein are well suited to separating samples containing species capable of reversibly interacting with boron.

The methods disclosed herein may be used for detecting markers linked to diseases and conditions where the markers contain functional groups that are capable of reversible interaction with the boronic acid groups present in the gel. Markers which may be detected by the methods of the present invention include disease linked carbohydrates in the blood which can be indicative for example of cancer. Other cancer markers include the CA-125 antigen and heptasaccharide markers. Glycated proteins, including early stage glycated proteins can be indicative of diseases, conditions or biological processes including ageing, diabetes, Alzheimer's disease. Polyhydric species such as carbohydrates and posttranslationally modified peptides can also be markers for microbial infections.

### EXAMPLES

In the examples below, it is shown that the incorporation of specialised carbohydrate affinity ligand methacrylamido phenylboronic acid in polyacrylamide gels for fluorophore-assisted carbohydrate electrophoresis greatly improved the effective separation of saccharides that show similar mobilities in standard electrophoresis. Polyacrylamide gel electrophoresis using methacrylamido phenylboronic acid in low loading (typically 0.5-1% dry weight) was unequivocally shown to alter retention of labelled saccharides depending on their boronate affinity. While conventional fluorophore-assisted carbohydrate electrophoresis of 2-aminoacridone labelled glucose oligomers showed an inverted parabolic migration, an undesired trait of small oligosaccharides labelled with this neutral fluorophore, boron affinity saccharide electrophoresis separation of these carbohydrates completely restored their predicted running order, based on their charge/mass ratio, and resulted in improved separation of the analyte saccharides. These results exemplify boron affinity saccharide electrophoresis as an important new technique for analysing polyhydric species such as carbohydrates and sugar-containing molecules.

In the examples below, it is demonstrated that the incorporation of specialized carbohydrate affinity ligand methacrylamido phenylboronic acid (MPBA) in polyacrylamide gels for SDS-PAGE analysis of post-translationally modified proteins shows effective detection and separation of non-enzymatic glycosylated proteins and unmodified proteins. While conventional SDS-PAGE analysis could not distinguish between glycated and unglycated proteins, polyacrylamide gel electrophoresis using MPBA in low loading showed dramatic retention of δ-gluconolactone modified recombinant proteins fused with an N-terminal histidine affinity tag, while the mobility of the unmodified protein remained unchanged. In addition to gluconoylated proteins also lactosyl β-Lactoglobulin conjugates could be identified, indicating that this method is highly selective for early glycation products. Phosphorylated and glycosylated proteins also showed altered retention in the MPBA incorporated gels albeit to a lesser extent compared to the linear saccharide containing early glycation products. These results demonstrate that the methods of the present invention are an important new tool for the detection and the design of inhibitors of early glycation products in recombinant protein production, ageing, diabetes, cardiovascular and Alzheimer's disease, and for detecting other post-translational modification of polypeptides.

### Resolving Carbohydrates

### Materials and Methods

### Synthesis and Analysis of Methacrylamido Phenylboronic Acid (MPBA)

### Solvents and Reagents

The solvents and reagents that were used throughout this project were reagent grade unless otherwise stated and were purchased from Acros Organics (Geel, Belgium), Alfa Aesar (Karlsruhe, Germany), Fisher Scientific UK (Loughborough, UK), Frontier Scientific Europe (Carnforth, UK), Sigma-Aldrich Company (St. Louis, Mo., USA), and were used without further purification.

### Infrared Spectra

Infrared spectra were recorded on a Perkin Elmer Spectrum RX spectrometer (Perkin Elmer, Waltham, Mass., USA) between 4400 and 450 cm⁻¹. Samples were either evaporated from CHCl₃ on a NaCl disc (neat) or mixed with KBr in a mortar and pressed into a KBr pellet (KBr). All vibrations (ν) are given in cm⁻¹.

### NMR Spectra

NMR spectra were run in either chloroform-d or methanol-d₄. A Bruker AVANCE 300 was used to acquire the NMR spectra, ¹H NMR spectra were recorded at 300 MHz, ¹¹B{¹H} NMR spectra at 96 MHz and ¹³C{¹H} NMR spectra at 76 MHz. Chemical shifts (δ) are expressed in parts per million and are reported relative to the residual solvent peak or to tetramethylsilane as an internal standard in ¹H and ¹³C{¹H} NMR spectra, boron trifluoride diethyl etherate as an external standard in ¹¹B{¹H} NMR spectra. The multiplicities and general assignments of the spectroscopic data are denoted as: Singlet (s), doublet (d), triplet (t), quartet (q), quintet (quin), doublet of doublets (dd), doublet of triplets (dt), triplet of triplets (tt), unresolved multiplet (m), broad (br) and aryl (Ar). Coupling constants (J) are expressed in Hz.

### Mass Spectrometry Analysis

For all of the mass spectra used in this report, a micrOTOF ESI-TOF mass spectrometer (Bruker Daltonik GmbH, Bremen, Germany) was used. The spectrometer was coupled to an Agilent Technologies 1200 LC system (Agilent Technologies, Santa Clara, Calif., USA). Ten microliters of sample was injected into a 30:70 flow of water/acetonitrile at 0.3 mL/min to the mass spectrometer. The nebulising gas used was nitrogen, which was applied at a pressure of 1 bar. Nitrogen was also used as a drying gas, supplied at a flow rate of 8 L/min and a temperature of 2001 C. Positive ion mode was used with a corresponding capillary voltage of −4000 V and only full scan data was acquired. Negative ion mode was used with a corresponding capillary voltage of +4000 V and only full scan data was acquired. In each acquisition 10 mL of 5 mM sodium formate clusters was injected before the sample. The sodium formate was there to act as a calibrant over the mass range 50-1500 m/z. Data acquisition and automated processing were controlled via Compass Open Access 1.2 software. The observed mass and isotope pattern perfectly matched the corresponding theoretical values as calculated from the expected elemental formula. These calculations were carried out using the Bruker data processing software, DataAnalysis 3.4.

### Synthesis of the Protected Monomer of MPBA (Compound 3 in FIG. 2; Referred to Herein as “Compound 3”)

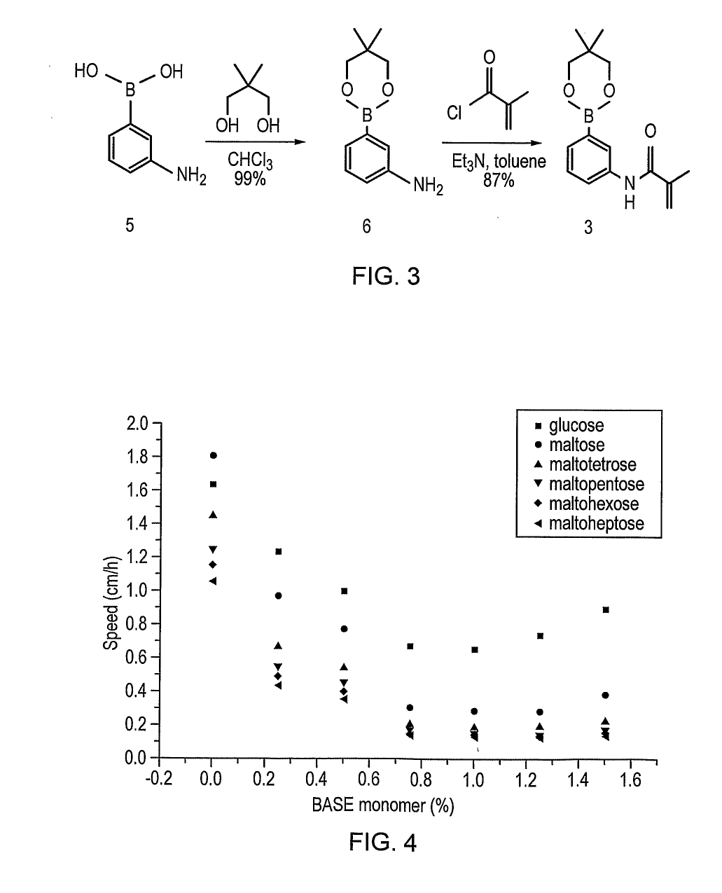

### Step 1: Protection of 3-aminophenylboronic acid, Synthesis of 3-(5,5-dimethyl-1,3,2-dioxaborinin-2-yl)benzeneamine (Compound 6; FIG. 3)

3-Aminophenylboronic acid (compound 5 (FIG. 3), monohydrate form, 0.419 g, 2.7 mmol) was stirred with chloroform (30 mL). 2,2-Dimethylpropanediol (0.281 g, 2.7 mmol) was added and the resulting suspension stirred until complete dissolution, the solution was filtered and dried in vacuo to give compound 6 (3-(5,5-dimethyl-1,3,2-dioxaborinin-2-yl)benzeneamine, see FIG. 3) as a white solid (0.550 g, 2.67 mmol, 99% yield). IR (ν, neat, cm⁻¹) 3442, 3360, 2959, 2878, 1581, 1482, 1445, 1325, 1247, 1128, 708 and 524. ¹H NMR (δ; 300 MHz; CDCl₃) 0.94 (6H, s, 2×CH₃), 3.68 (4H, s, 2×OCH₂) 6.68 (1H, ddd, J57.5, 2.7 and 1.5, Ar CH), 7.11 (3H, m, Ar CH). ¹³C{¹H} NMR (δ; 75 MHz; CDCl₃) 22.3 (2×CH₃), 32.3 (Cq), 72.7 (2×OCH₂), 118.0 (Ar CH), 120.8 (Ar CH), 124.6 (Ar CH), 129.0 (Ar CH), 146.0 (Ar C—N), (C—B not detected). ¹¹B{¹H} NMR (δ; 96 MHz; CDCl₃) 28.0 bs. MS (ESI, positive, CH₃OH) found m/z 206.1351, C₁₁H₁₇¹¹BNO₂ requires m/z 206.1352. See also [6, 7].

Step 2: Addition of methacryloyl chloride, synthesis of compound 3. Compound 6 (see FIG. 3) (0.49 g, 2.39 mmol), triethylamine (336 mL, 2.39 mmol) and toluene (30 mL) were cooled to 0° C. and stirred under nitrogen. A solution of methacryloyl chloride (234 mL, 2.39 mmol) in toluene (10 mL) was added slowly (0.2 mL/min) at 0° C. The solution was allowed to warm to room temperature and stirred for further 30 min. Water (30 mL) was added, the organic layer was separated, dried over magnesium sulphate, filtered, and dried in vacuo (<30° C.). Repeated (three times) dissolution in and evaporation of dichloromethane in vacuo (<30° C.) insured complete removal residual toluene. MPBA (compound 3, FIG. 3) was obtained as white solid (0.572 g, 2.09 mmol, 87% yield). IR (ν, neat, cm⁻¹) 3311, 2961, 2886, 1662, 1626, 1539, 1482, 1427, 1318, 1248, 1128 and 705. ¹H NMR (δ; 300 MHz; CDCl₃) 0.94 (6H, s, 2×CH₂), 3.68 (4H, s, 2×OCH₂), 5.35 (1H, m, J=0.6, C—CH), 5.70 (1H, m, J=0.6, C—CH), 7.26 (1H, t, J=7.8, Ar CH), 7.47 (1H, dt, J=7.5 and 1.2, Ar CH), 7.74 (1H, dm, J=1.2), 7.82 (1H, ddd, J=8.0, 2.4 and 1.2, Ar CH). ¹³C{H} NMR (δ; 75 MHz; CDCl₃) 19.1 (methacryl CH₂) 22.3 (2×CH₂), 32.3 (Cq), 72.7 (2×OCH₂), 120.1 (methacryl CH₂), 123.0 (Ar CH), 125.5 (Ar CH), 128.8 (Ar CH), 130.2 (Ar CH), 137.7 (methacryl Cq), 141.3 (Ar C—N), 166.9 (CO amide) (C—B not detected). ¹¹B{¹H} NMR (δ; 96 MHz; CDCl₃) 25.9 bs. MS (ESI, positive, CH₃OH) found m/z 296.1434, C₁₅H₂₀¹¹BNO₃Na⁺ requires m/z 296.1428. Compound hydrolyses during analysis to corresponding acid: (acid1H1) found m/z 206.1008, C₁₀H₁₃¹¹BNO₃⁻ requires m/z 206.0983 and (acid+Na⁺) found m/z 228.0813, C₁₀H₁₂¹¹BNO₃Na⁺ requires m/z 228.0802. MS (ESI, negative CH₃OH) shows only corresponding acid (acid —H)⁻ found m/z 204.0825, C₁₀H₁₁¹¹BNO₃⁻ requires m/z 204.0837.

Fluorophore Labelling of Saccharides with AMAC

Mono- and oligosaccharides were derivatised with AMAC (Sigma-Aldrich) as described by Gao and Lehrman [6]. In brief: dried saccharides (˜20 nmol) were dissolved in 5 mL AMAC solution (0.1M AMAC in DMSO (containing 15% v/v acetic acid (Fisher Chemicals)) and 5 mL of freshly prepared sodium cyanoborohydride solution (1M sodium cyanoborohydride (Sigma-Aldrich) in DMSO, AnalaR, Poole, Dorset, UK)), mixed well, briefly centrifuged and incubated at 37° C. for 16 h.

### FACE, Gel Imaging and Data Analysis

Monosaccharide profiling polyacrylamide gels were prepared as described previously (see detailed description by Gao and Lehrman [10]). Resolving gels were polymerised in the absence or presence of MPBA (compound 3, see FIG. 2) in concentrations ranging from 0 to 2%. Samples of AMAC labelled saccharides (5 mL in volume) were loaded onto gels (height: 100 mm×width: 100 mm×thickness: 0.75 mm) with single concentrations of polyacrylamide (ranging from 10 to 40%) in Tris-boric acid electrophoresis buffer (0.12M Tris Base Ultrapure (Melford Laboratories, Chelsworth, UK), 0.1M glycine (Melford Laboratories) and 0.1M boric acid (Sigma-Aldrich), as described previously [10]), with pHs ranging from 7.5 to 9.0. Labelled saccharide samples were electrophoresed at 41° C. for 2-8 h, depending on their migration time, at a constant current of 20 mA (with voltages in the range of 90-300 V). Saccharide separation results were visualised and digitally captured on a AlphaImager 3400 UV transilluminator (Alpha Innotech, San Leandro, Calif., USA). Inverse images of the saccharide gel profiles were subsequently processed using Adobe Photoshop. Mobilities of the saccharides were expressed as a function of migration distances versus time.

### Producing Electrophoresis Gels Templated to Bind Specific Species

0.2% w/v of fructose and 0.2% w/v of methacrylamido phenylboronic acid (MPBA) was dissolved in 8% acrylamide solution (from 40% stock solution of acrylamide:bis-acrylamide, 29:1; Fisher Scientific, Fair Lawn, N.J., USA) in 40 mM Tris buffer at pH 8.8 solution prior to polymerisation. The gel was cast in a gel cassette (height 100 mm×width 100 mm×thickness 0.75 mm; Invitrogen, Carlsbad Calif., USA) using ammonium persulfate (APS) and tetramethylethylenediamine (TEMED) as radical initiators. Saturated butanol was carefully added to level the gel. Following polymerisation of the resolving gel, butanol was rinsed off and stacking gel, containing no boronic acid and prepared with 10% acrylamide (from 40% stock solution of acrylamide:bis-acrylamide, 29:1; Fisher Scientific) in 10 mM Tris buffer pH 6.8 was cast on top the resolving gel. A comb was inserted before this solution polymerises to create sample wells. Protein samples in sample buffer were applied to the stacking gel and electrophoresed at 60 mA for 60 min in glycine buffer (25 mM Tris pH 8.3, 250 mM glycine and 0.1% SDS) at room temperature.

### Results

The present inventors thought that inclusion of receptors that reversibly interact with saccharides would advantageously affect retention characteristics, especially if the receptor displays differential interactions with diverse saccharides. The chosen receptor would need to be (i) easily incorporated into electrophoresis gels since covalent linking would prevent receptor leaching; (ii) able to differentially bind analyte saccharides and (iii) be tolerant to water. Phenyl boronic acids have the capacity to function as saccharide receptors in aqueous solution, attested by the many sensory systems reported [11-14]. They have been shown to form cyclic boronic esters with various carbohydrates under equilibrium conditions, via reversible covalent interactions in aqueous media, as is illustrated in FIG. 1. Boronic acids display differing binding affinities, independent of size/charge ratios and for this quality boronic acid-functionalised gels have been utilised as a stationary phase for the analysis and separation of mono-, oligosaccharides and oligonucleosides by column chromatography [15]. Electrophoresis gels are commonly a hydrogel assembled by copolymerising acrylamide (compound 1, FIG. 2) with the cross-linker methylene bisacrylamide (compound 2, FIG. 2). Acrylamide gels incorporating boronate were easily prepared by adding a small percentage of MPBA (compound 3, FIG. 2) to the electrophoresis gel preparation solution prior to polymerisation. Compound 3 was synthesised from compounds 5 (3-aminophenylboronic acid) and 6 (3-(5,5-dimethyl-1,3,2-dioxaborinin-2-yl)benzeneamine, FIG. 3; for synthesis details see Experimental Section). To ensure any saccharide retention effects observed for boronate-containing gels were not due to the introduction of either the concomitant methyl and phenyl groups of the MPBA gels with N-phenylmethacrylamide (compound 4, FIG. 2) were also cast for comparison of steric effects.

### Example 1

### Separation of Glucose Oligomers

FACE was used to analyse the effect of gel-incorporated boronate on the separation of glucose oligomers. The most commonly used derivatives for the fluorometric detection of mono- and oligosaccharides in FACE are 8-aminonaphthalene-1,3,6-trisulphonic acid and AMAC. The trisulphate moiety of 8-aminonaphthalene-1,3,6-trisulphonic acid provides three negative charges to the labelled sugars and contributes to the electromobility of the sugars in FACE analysis. The fact that the AMAC fluorophore has no ionic charge in commonly used electrophoretic buffers makes it a suitable derivative for the separation of neutral and acidic saccharides [34], which more accurately reflects their charge/mass ratio. The slower migrating neutral AMAC labelled oligosaccharides can also be separated as a function of molecular size when borate ions are present in the electrophoresis buffer. However, inverted migration patterns have been observed in the separation of small AMAC-labelled oligosaccharides [7-9], questioning the suitability of AMAC labelling for the separation of oligosaccharide mixtures [35].

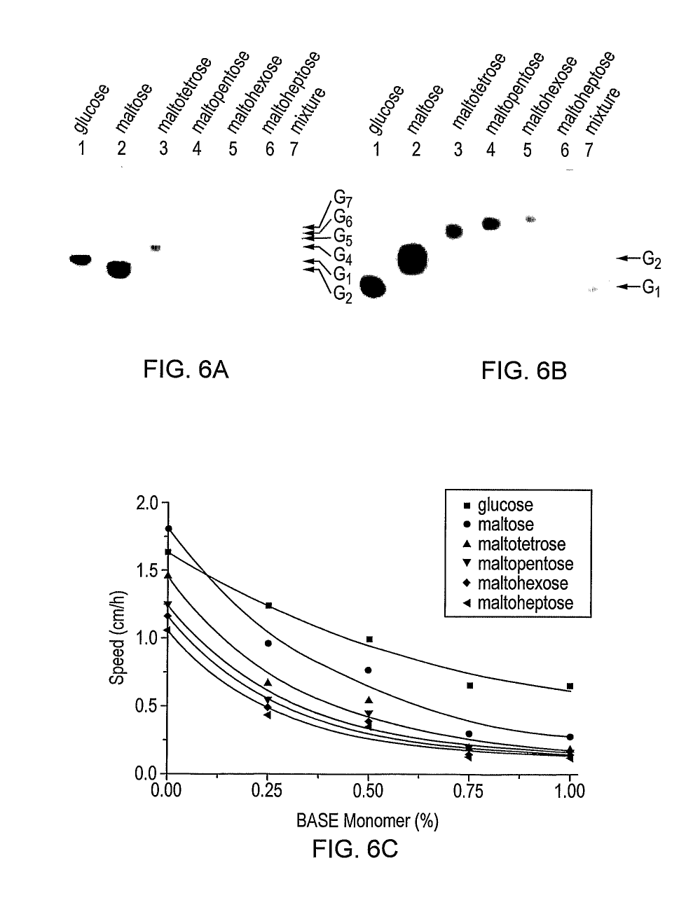

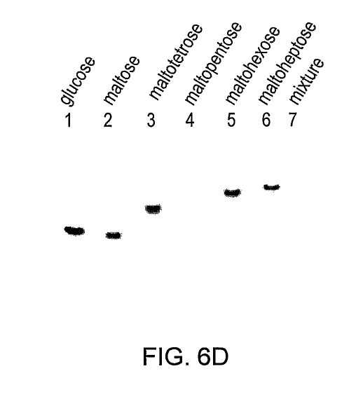

FIG. 6A (lanes 1-6) depicts the migration profile of AMAC-labelled glucose oligomers (G₁, G₂, G₄, G₅, G₆ and G₇, individual sugars and a saccharide mixture) using a standard FACE protocol, in the presence of borate (see Experimental section). The electrophoretic mobilities of the longer AMAC-labelled oligosaccharides (G₂-G₇) are related largely to their molecular size (Mᵣ). Under the same experimental conditions, however, fluorophore-labelled glucose (G₁) shows an inversed mobility migrating at a higher relative molecular size/charge position than maltose (G₂, see also the relative mobility (speed) analysis in FIG. 6C). This inverted migration pattern, also observed by others [7-9], is thought to be caused by the differential interaction of the smaller oligosaccharides with borate ions, present in the electrophoresis buffer [7]. When MPBA is incorporated in the polyacrylamide gel matrix, by co-polymerisation, the migration pattern of the glucose oligomers changes significantly. A defined ladder-type separation as a function of oligosaccharide length and MPBA incorporation is observed for all the AMAC-labelled glucose oligomers (FIG. 6B), with glucose and maltose now relatively positioned based on their molecular weight. Most notably, with the boronate derivatised gels the separation of maltose and glucose has dramatically improved. Whilst an increased difference in the mobility of maltose and maltotetrose was noted, the migration pattern and the relative positions of the longer glucose oligomers (G₄-G₇) remain virtually unchanged. To ensure any saccharide retention effects observed for boronate-containing gels were not due to steric effects caused by the introduction of either the concomitant methyl and phenyl groups of the MPBA, the results were compared with gels derivatised with N-phenylmethacrylamide (compound 4, FIG. 2). FIG. 6D shows that the AMAC-labelled glucose oligomer separation profile with N-phenylmethacrylamide derivatised polyacrylamide gels is identical to that of a normal FACE analysis of these labelled saccharides, including the typical inverted migration pattern.

FIG. 6C shows a plot of the exponential retention increase (speed decrease) of each of the six analyte saccharides as a function of MPBA incorporation (up to 1.0%). Data were collected up to the solubility limit of MPBA (1.5%), but gels that included 1.0% ran exponentially slower. The extended running times necessary for the gels that displayed extreme saccharide retention, resulted in slight physical defects in the gels, thus measurements taken were not so reliable. As already pointed out in the absence of MPBA, maltose and glucose suffer from an inversion in expected running order, which is a major obstacle for the use of neutral labels in the FACE technique. It is noteworthy that incorporation of just 0.25% of MPBA, utilising the boron affinity saccharide electrophoresis (BASE) method of the present invention, results in restoration of the predicted saccharide running order. At 0.5% the best separation of analyte saccharides could be achieved.

The system was also tested for linearity between mobility (mm/h) and logarithms of molecular mass (Mr) of the glucose oligomers. Linearity was observed throughout the molecular mass range from G₁-G₇ at MPBA monomer concentrations between 0.25 and 0.5% (with correlation coefficients (R²) of 0.9986 and 0.9995, respectively). At 1.0% the gel system had become almost impermeable to the larger oligosaccharides and, although linearity of the separation is lost at this MPBA monomer concentration, it provided excellent separation between glucose and maltose and the G₄-G₇ glucose oligomers.

### Example 2

### Separation of Mono- and Disaccharides

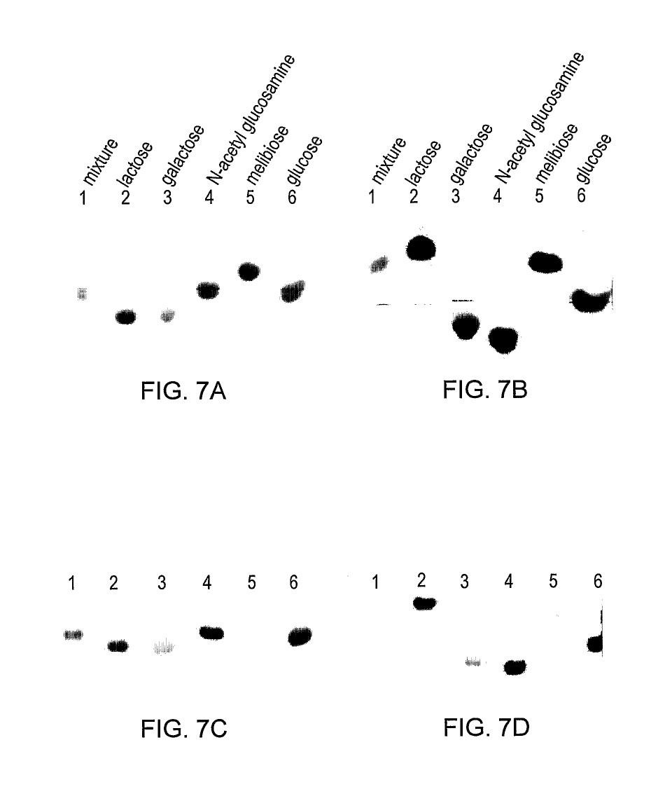

The performance of the method was further evaluated by the separation of a series of AMAC-labelled mono- and disaccharides. The AMAC-labelled monosaccharides glucose (Glc), galactose (Gal) and N-acetyl glucosamine (GlcNAc) were chosen because they have been shown to be the most difficult to separate in FACE analysis [10] and compared their electrophoretic mobility with those of disaccharides lactose and melibiose. FIG. 7A shows that AMAC-labelled monosaccharides Glc, Gal and GlcNAc can be separated using the conventional FACE analysis, as reported previously, albeit very difficult to distinguish between the Glc and GlcNAc bands in the saccharide mix (lane 1). The disaccharides included in this FACE profile also reveal that although this method can distinguish between acidic and neutral oligosaccharides, it is not suitable for separation based on mass characteristics. As can be seen in FIG. 7A, monosaccharide Gal cannot be separated from Gal-containing disaccharide lactose and moves faster than its other monosaccharide component Glc. Interestingly, disaccharide lactose (Gal(β-4)Glc) migrates significantly faster than its structural isomer melibiose (Gal(α1-6)Glc), confirming the finding by other studies that in addition to charge, structural elements such as linkage position, and linkage anomericity contribute to glycan mobility [36]. The larger hydrodynamic radius and flexibility associated with the α1-6 linkage could possibly explain the reduced electrophoretic mobility of melibiose, compared with lactose.

Separation of AMAC-labelled monosaccharides Glc, Gal and GlcNAc is greatly improved using the methods of the present invention as can be seen in FIG. 7B, with GlcNAc showing the highest mobility and Glc the lowest, reflecting their relative affinity for the MPBA incorporated in the gel. In accordance with the analysis of glucose oligomers (FIG. 6), the monosaccharides have been separated from disaccharides in FIG. 7B, thereby reflecting the ‘true’ charge/mass characteristics of the saccharides.

So far all sugar separations described in this paper were performed at pH 8.3, close to the pKa value of PBA (8.8). To investigate the effect of deprotonation/ionisation So far all sugar separations described in this paper were performed at pH 8.3, close to the pKa value of PBA (8.8). To investigate the effect of deprotonation/ionisation of MPBA in the gel on the separation of saccharides, the experiment described in FIGS. 7A and B were repeated using electrophoresis buffer with pH 7.5 and 9.0. At pH 7.5 the carbohydrate separation pattern observed is identical to that seen in the pH 8.3 gels; however, the low electro-osmotic flow resulted in band broadening and very long migration times (48 h) in both normal and MPBAincorporated gels. In a normal gel run at pH 9.0 (FIG. 7C) the separation of AMAC-labelled saccharides is significantly reduced compared with electrophoresis at pH 8.3. Although a similar effect can be observed for the monosaccharides in the MPBA-containing gels, at the same time the separation of mono- and disaccharides is preserved

### Example 3

### Speed of Saccharide vs MPBA Content

Speed was determined as follows: glucose monomers and oligomers glucose, maltose, maltotetraose, maltopentaose, maltohexaose and maltoheptaose were electrophoresed on gels with varying compound 3 content (0, 0.25, 0.50, 0.75, 1.00, 1.25 & 1.50%) and visualised (under UV) at recorded time intervals. Distance (mm)/time(h) was plotted for each saccharide on each gel. The graph shown in FIG. 4 shows speed/concentration data up to 1.5% MPBA. Non-exponential behaviour was observed beyond 1.0% MPBA inclusion. FIG. 5 shows en exponential relationship for 0.25 and 0.5 MPBA inclusion.

### Detecting Posttranslational Modification

### Materials and Methods

### Glyconoylation of Sbi-III-IV.

Sbi-III-IV was freshly expressed and purified as described previously [30] and incubated with 100 mM freshly prepared D-(+)-Gluconic acid δ-lactone (Sigma Aldrich) in a water bath at 37° C. for 15 min-16 hours.

### Preparation of Methacrylamido Phenylboronic Acid (MPBA)

### Polyacrylamide Gels

Protected methacrylamide phenylboronic acid (MPBA or MBA, FIG. 2, compound 3) was synthesized as described by D'Hooge et al [3]. Polyacrylamide electrophoresis gels used for protein separation are commonly a hydrogel assembled by copolymerising acrylamide with the cross-linker methylene bisacrylamide. Acrylamide gels incorporating boronate were easily prepared by adding a small percentage (0-1%) of MPBA to the electrophoresis gel preparation solution prior to polymerisation. Polyacrylamide resolving gels were polymerised in the absence or presence of MPBA (compound 3, FIG. 2) in concentrations ranging from 0-1% by mixing MPBA powder (0-1%) with a 15% acrylamide solution (from 40% stock solution of acrylamide:bis-acrylamide, 29:1; Fisher Scientific, Fair Lawn, N.J., USA) in 40 mM Tris buffer at pH 8.8 and cast in a gel casting cassette (height: 100 mm×width: 100 mm×thickness: 0.75 mm; Invitrogen, Carlsbad Calif., USA). After polymerisation of the resolving gel, using 10% Ammonium persulfate (APS, Sigma Aldrich, St Louis, Mo., USA) and N,N,N′,N′-tetramethylethylene-diamine (TEMED, Sigma Aldrich) the stacking gel, containing no boronic acid, and was prepared with 10% acrylamide (from 40% stock solution of acrylamide:bis-acrylamide, 29:1; Fisher Scientific) in 10 mM Tris buffer pH 6.5 and cast on top the resolving gel. The protein samples where applied to the stacking gel in sample buffer (2% w/v SDS, 2 mM Dithiothreitol, 15% glycerol, 100 mM Tris pH 6.8 and bromophenol blue) and gels were electrophoresed at 50 mA for 30-45 min in glycine buffer (25 mM Tris pH 8.3, 250 mM glycine and 0.1% SDS) at room temperature or a 4° C.

### Mass Spectrometry Analysis of Glyconoylated Proteins

For all of the Mass Spectra used in this report, a micrOTOF electrospray time-of-flight (ESI-TOF) mass spectrometer (Bruker Daltonik GmbH, Bremen Germany) was used. The spectrometer was coupled to an Agilent Technologies 1200 LC system (Agilent Technologies, Santa Clara, Calif., USA). 10 μL of sample was injected into a 30:70 flow of water/acetonitrile and formic acid at 0.3 mL/min to the mass spectrometer. The nebulising gas used was nitrogen, which was applied at a pressure of 1 bar. Nitrogen was also used as a drying gas, supplied at a flow rate of 8 L/min and a temperature of 200° C. Positive ion mode was used with a corresponding capillary voltage of −4000 V and only full scan data was acquired. Negative ion mode was used with a corresponding capillary voltage of +4000 V and only full scan data was acquired. In each acquisition 10 μL of 5 mM sodium formate clusters was injected before the sample. The sodium formate was there to act as a calibrant over the mass range 50-1500 m/z. Data acquisition and automated processing were controlled via Compass Open Access 1.2 software. The observed mass and isotope pattern perfectly matched the corresponding theoretical values as calculated from the expected elemental formula. These calculations were carried out using the Bruker data processing software, DataAnalysis 3.4.

### Example 4

### SDS PAGE Analysis of Glycosylated Proteins

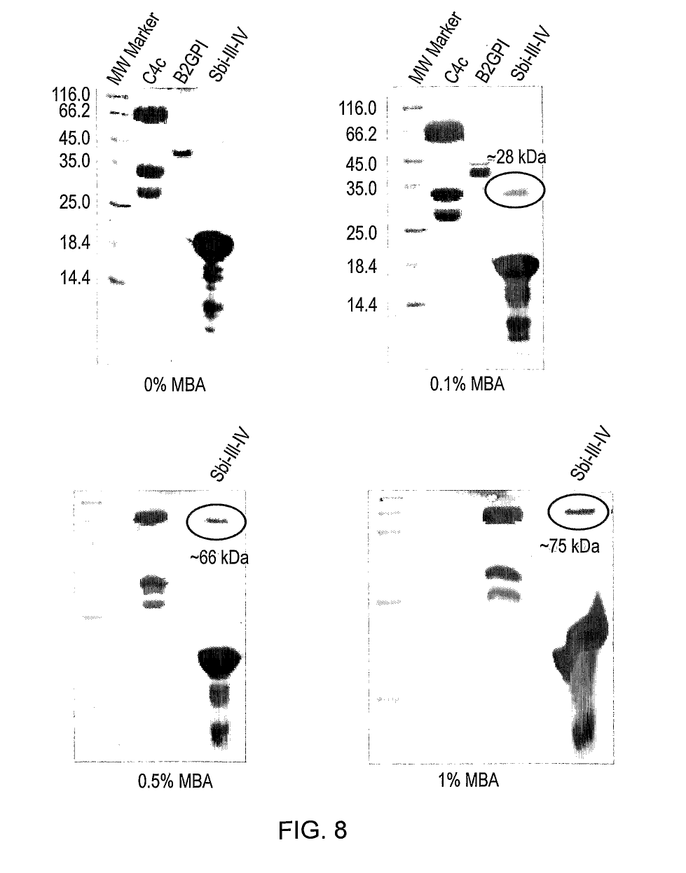

The SDS-PAGE analysis of glycoslylated proteins C4c and beta 2 Glycoprotein I (B2GPI) in comparison with non-glycosylated recombinant protein construct Sbi-III-IV was examined. FIG. 8 shows that with increasing concentrations of MPBA (MBA), a new protein band appears in the Sbi-III-IV lane. The apparent molecular weight of the new protein band increases with increasing MBA concentration. In addition, a slight shift can be observed in the C4c and (B2GPI).

### Example 5

### Modification of Sbi-III-IV in His-tag

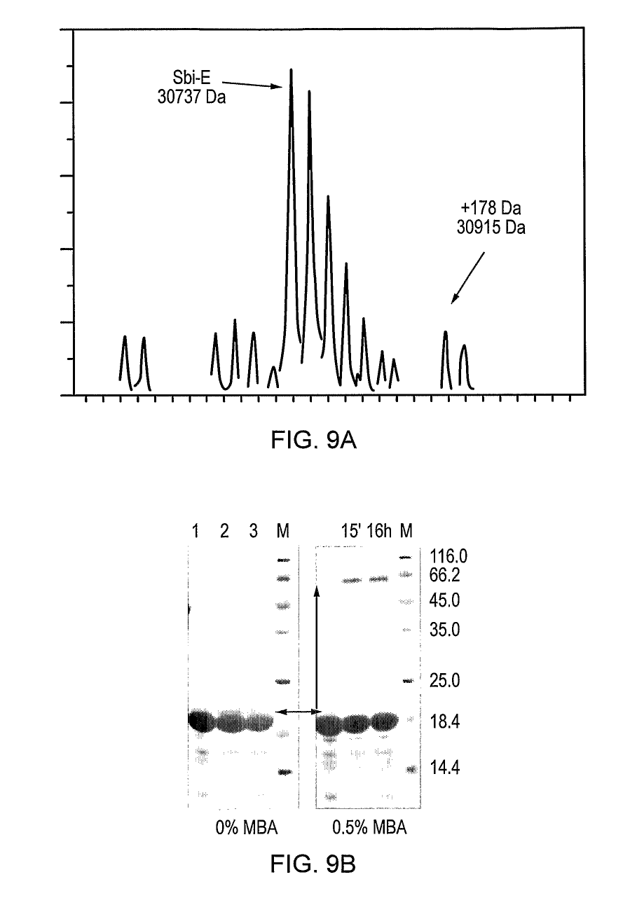

FIG. 9B compares a normal SDS-PAGE profile with a MPBA (MBA) gel according to the present invention, both showing the migration profile of the mixture, analysed by mass spectrometry in FIG. 9A, of unmodified and gluconolylated Sbi-III-IV (lane 1) after expression in E. coli and purification using nickel-ion chelating chromatography as described previously [30]. Lanes 2 and 3 show migration profiles of Sbi-III-IV with exogenously added δ-gluconolactone (100 mM) and incubated for 15 minutes and 16 hours, respectively. Under normal SDS-PAGE conditions the gluconoylated Sbi-III-IV fraction in the freshly expressed and purified protein cannot be distinguished from the unglycated protein, even when large amounts of protein are loaded onto the gel as shown in FIG. 9B. In the lanes containing Sbi-III-IV incubated with added δ-gluconolactone a faint shadow band appears just above the 17 kDa Sbi-III-IV that may hint the presence of the modified protein.

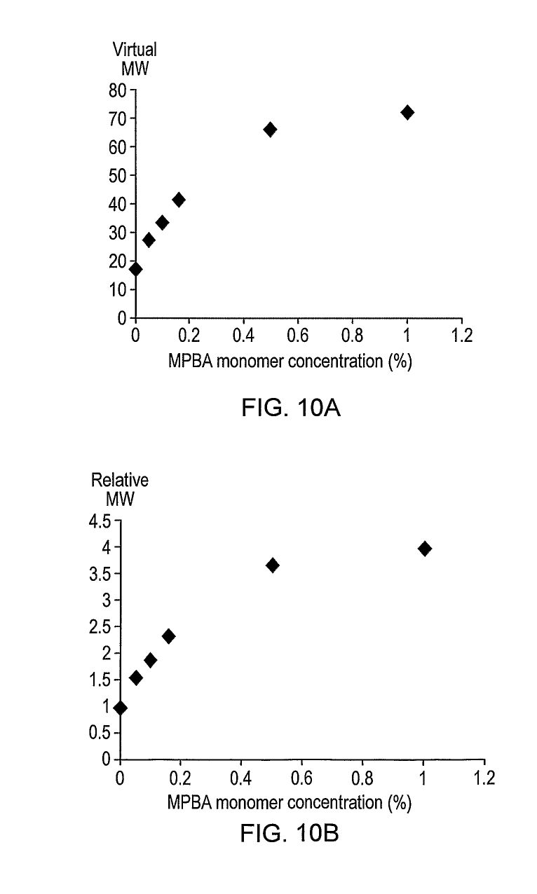

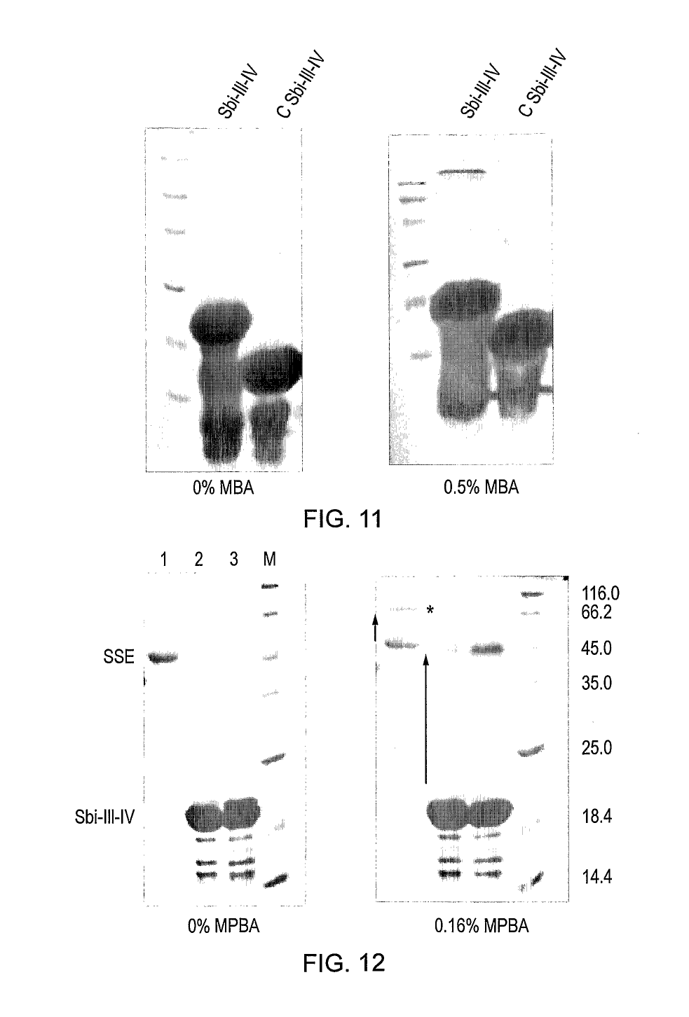

In contrast, the migration profile in the MPBA incorporated gel shows a dramatic separation of the modified and unmodified proteins, with the boronate affinity greatly affecting the mobility of the gluconoylated Sbi-IV, retaining it at a position expected for a protein quadruple the expected molecular size (FIG. 9B). The retention of gluconoylated Sbi-III-IV is strongly correlated with the concentration of MPBA incorporated in the gel (0; 0.05; 0.1; 0.16; 0.5 and 1%), as is shown in FIGS. 10A and 10B, with the highest degree of retention observed in the 1% MPBA gel. FIG. 11 shows that the glyconyolation site is indeed located in the N-terminal histidine tag. Gel profiles were compared in the absence (left) and presence of MPBA (0.5%, right) of intact recombinant protein construct Sbi-III-IV and Sbi-III-IV with cleaved histidine tag (C Sbi-III-IV). The additional high molecular weight band in the 0.5% MBA gel appears as a low molecular weight band after cleavage, indicating that the modification that is retained in the MPBA gel is specific for Sbi-III-IV and is located in the His-tag. In FIG. 12 it is shown that the detection and separation of δ-gluconolactone modified and unmodified protein is also achieved in other recombinantly expressed proteins in which this specific modification was detected using mass spectrometry. Even at concentrations as low as 0.16% of incorporated MPBA, gluconylated proteins are retained in the gel at a virtual molecular size of twice their actual size. SSE is shown in lane 1, and Sbi-III-IV is lanes 2 and 3.

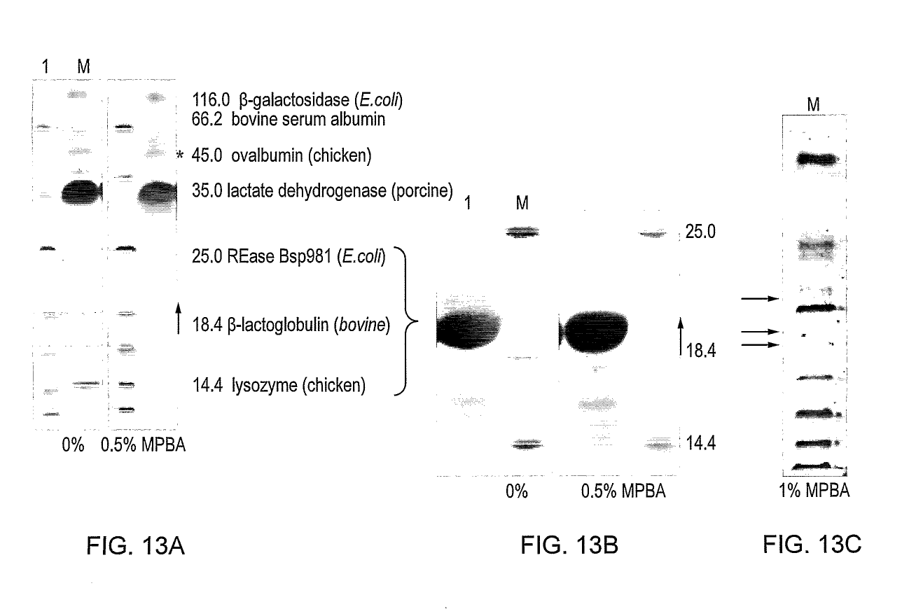

FIG. 13A highlights the fact that the molecular weight markers used in the gels presented in FIG. 9B are not significantly affected in their mobility by the presence of the MPBA monomer in the gel, ensuring that any saccharide retention effects observed for boronate containing gels were not due to the introduction of either the concomitant methyl and phenyl groups of the MPBA. Molecular weight markers bovine β-lactoglobulin and to a lesser degree ovalbumine, however are two exceptions. A significant retention of the bovine β-lactoglobulin protein band can be observed when comparing migration profiles of control gels with 0.5% MPBA gels that were electrophoresed in the same gel tank, at the same time. β-lactoglobulin clearly moves from a position below the unmodified Sbi-III-IV band in the control gel, to a location higher that the recombinant protein band, whereas the positions of the two adjacent marker proteins (E. coli restriction endonuclease Bsp981 (25.0 kDa) and chicken lysozyme (14.4 kDa) remain unchanged (see enlarged view, FIG. 13B). The boronate affinity based retention effect on the mobility of bovine β-lactoglobulin becomes more evident in the 1% MPBA gel, shown in FIG. 13C where three β-lactoglobulin bands can be observed, with the highest band close to the 25 kDa marker. Similar to the retention of gluconolylated Sbi-III-IV and SSE, the retention of β-lactoglobulin relative to the other molecular weight marker proteins can be explained by non-enzymatic glycosylation. Under conditions of mild heat treatment applied to milk, lactosylation of β-lactoglobulin can be commonly observed resulting in at least two different modified species [37-39]. The lactosylated lysine residues in these modified β-lactoglobulin represent products at an early stage of glycation since reactions subsequent to the Amadori rearrangements are suppressed or slowed in milk [37].

### Example 6

### Detection of Glycosylated and Phosphorylated Proteins

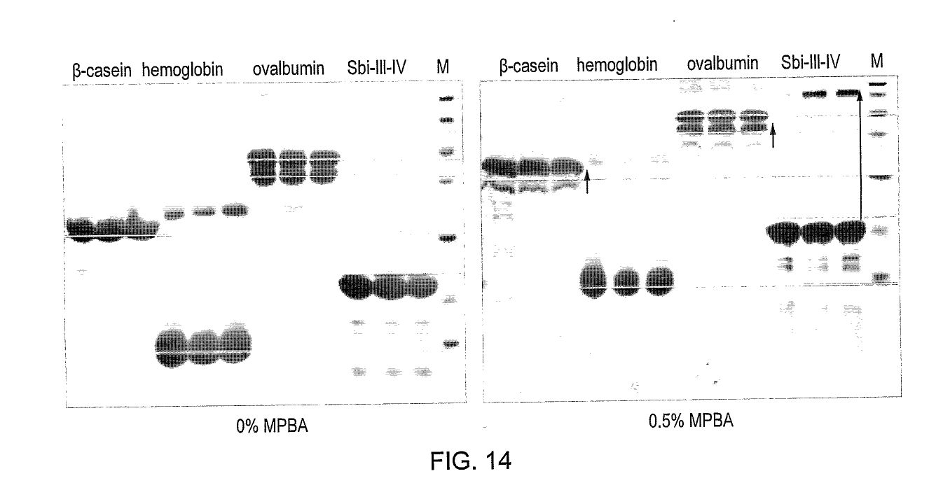

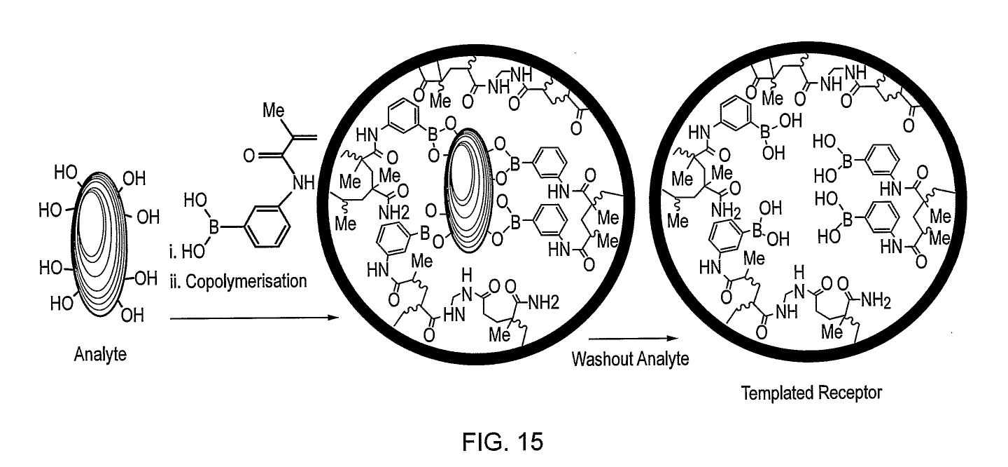

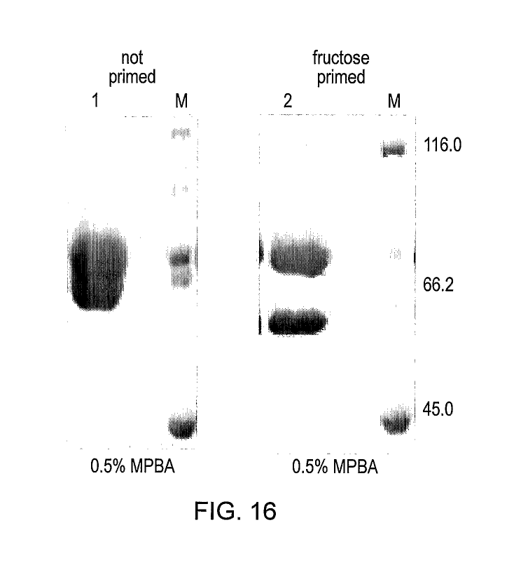

In this example, the effect on mobility of other posttranslational modifications, including phosphorylation, glycosylation and combinations thereof, is considered. In FIG. 14 are shown the SDS-PAGE relative mobilities of β-casein (phosphorylated/not glycosylated), human hemoglobin (not phosphorylated/not glycosylated) and chicken ovalbumin (phosphorylated/glycosylated). Small but significant shifts in relative mobility can be observed with β-casein and ovalbumin in the 0.5% MPBA gel, when compared with their positions in the control gel and those of non-glycosylated hemoglobin in both gels. Although these shifts in relative mobility are not as dramatic as seen in the gluconoylated Sbi-III-IV example, the results clearly indicate that the presence of boronate in SDS-PAGE could be used for the detection of post-translational glycosylation and phosphorylation.

With the improved separation of carbohydrates and absence of the inverted parabolic migration of small oligosaccharides, a major obstacle to the use of neutral labels in FACE, the methods of the present invention could become an important new technique for analysing carbohydrates and sugar-containing molecules, while retaining a separation that reflects the saccharide's ‘true’ charge/mass characteristics.

While the retention of glycosylated as well as phosphorylated proteins is affected by gel-incorporated boronate, the method proves to be a highly selective technique for the detection of early glycation products in proteins, including gluconoylation and lactosylation, suggesting that MPBA has a higher affinity for linear sugar adducts. These characteristics render the methods of the present invention ideal for the identification, estimation and separation of gluconoylation in recombinant protein expression. In addition, this technique will advance the study of spontaneous glycation processes in ageing, diabetes, cardiovascular and Alzheimer's disease by detecting known and new glycoxi-adducts, analyse potential inhibitors of the accumulation of AGEs and design new drugs that can remove these undesired adducts.

Any number of polyhydric species, such as DNA, RNA, glycoproteins and phosphoproteins can potentially be analysed by this technique, including those indicative of disease. We envisage the incorporation of boronic acids into electrophoresis gels has the potential to become routine in many analytical and biomedical laboratories adding an economical, reliable and robust dimension to existing analyses as well as leading to the development of new carbohydrate-based assays.

### Example 7

### Use of Template Molecules for Ligand-Specific Gel Templating

Experiments were carried out to validate the use of molecular imprinting techniques using the electrophoresis gels of the present invention. In this experiment, an electrophoresis gel was made by copolymerising a boronic acid species, a polymerisable linker and a template molecule, in this case, fructose. The fructose served as a template around which the gel formed, providing regions in the gel that are generally complementary to the size and shape of the fructose template.

This gel was then used in an experiment to compare how templating the gel with fructose affected the separation of fructosamine-HSA and unglycated HSA. FIG. 16 shows that the fructose templated gel dramatically improved the separation of the two species, with the fructosamine-HSA being able to interact with the gel by displacing the fructose template molecules because of their similar structures.

### REFERENCES

All publications, patents, and patent applications cited herein are hereby incorporated by reference in their entirety for all purposes.
[1] Boronic Acids—Preparation and Applications in Organic Synthesis and Medicine; Hall, D. G. (Ed); Wiley-VCH, Weinheim 2005
[2] Scrafton D. K. et al, J. Org. Chem., 2008, 73, 2871-2874
[3] D'Hooge F et al; Polymer, 2008, 49, 3362-3365
[4] Igloi G and Kossel H; Nucleic Acids Research, 1985, Vol 13 No 19, 6881-6898
[5] Gao et al., Anal. Lett. 2003, 36, 1281-1310.
[6] Jackson, Biochem. J. 1990, 270, 705-713.
[7] Calabro et al., Glycobiology 2000, 10, 273-281.
[8] Tawada et al., Glycobiology 2002, 12, 421-426.
[9] Mahoney, et al., Glycobiology 2001, 11, 1025-1033.
[10] Gao & Lehrman, Glycobiology 2003, 13, 1G-3G.
[11] James, in: Hall, D. G. (Ed.), Boronic Acids in Organic Synthesis and Chemical Biology, Wiley-VCH, Weinheim 2005, pp. 441-480.
[13] James et al., Boronic Acids in Saccharide Recognition. RSC Publishing, Cambridge, UK 2006, p. 200.
[14] James & Shinkai, Top. Curr. Chem. 2002, 218, 159-200.
[15] Schott, Angew. Chem. Int. Ed. Engl. 1972, 11, 824-825
[16] Gray C et al.; Tet. Lett, 2007, 48, 2683-2686
[17] Wells-Knecht, M. C., Thorpe, S. R., Baynes, J. W., Biochemistry 1995, 34, 15134-15141.
[18] Rahbar, S., Cell Biochem Biophys 2007, 48, 147-157.
[19] Unoki, H., Yamagishi, S., Curr Pharm Des 2008, 14, 987-989.
[20] Lindsay, R. M., Smith, W., Lee, W. K., Dominiczak, M. H., Baird, J. D., Clin Chim Acta 1997, 263, 239-247.
[21] Rahbar, S., Nadler, J. L., Clin Chim Acta 1999, 287, 123-130.
[22] Horbowicz, M., in: Ellis, R. H., Black, M., Murdoch, A. J., Hong, T. D. (Eds.), Basic and applied aspects of seed biology. Proceedings of the Fifth International Workshop on Seeds, Reading, UK 10-15 Sep. 1995, Kluwer Academic Publishers 1997.
[23] Lahuta, L. B., R. J., G., Zalewski, K., Hedley, C. L., Acta Physiol Plant 2007, 29, 8.
[24] Aon, J. C., Caimi, R. J., Taylor, A. H., Lu, Q., et al., Appl Environ Microbiol 2008, 74, 950-958.
[25] Geoghegan, K. F., Dixon, H. B., Rosner, P. J., Hoth, L. R., et al., Anal Biochem 1999, 267, 169-184.
[26] Yan, Z., Caldwell, G. W., McDonell, P. A., Jones, W. J., et al., Biochem Biophys Res Commun 1999, 259, 271-282.
[27] Yan, Z., Caldwell, G. W., McDonell, P. A., Biochem Biophys Res Commun 1999, 262, 793-800.
[28] Beranek, M., Drsata, J., Palicka, V., Mol Cell Biochem 2001, 218, 35-39.
[29] Kim, K. M., Yi, E. C., Baker, D., Zhang, K. Y., Acta Crystallogr D Biol Crystallogr 2001, 57, 759-762.
[30] Burman, J. D., Leung, E., Atkins, K. L., O'Seaghdha, M. N., et al., J Biol Chem 2008, 283, 17579-17593.
[31] Fu, M. X., Wells-Knecht, K. J., Blackledge, J. A., Lyons, T. J., et al., Diabetes 1994, 43, 676-683.
[32] Wells-Knecht, K. J., Zyzak, D. V., Litchfield, J. E., Thorpe, S. R., Baynes, J. W., Biochemistry 1995, 34, 3702-3709.
[33] Loske, C., Gerdemann, A., Schepl, W., Wycislo, M., et al., Eur J Biochem 2000, 267, 4171-4178.
[34] Jackson, Anal. Biochem. 1991, 196, 238-244
[35] Seyfried et al, Glycobiology 2005, 15, 303-312
[36] Masuoka and Hazen, Electrophoresis 2006, 27, 365-372
[37] Leonil, J., Molle, D., Fauquant, J., Maubois, J. L., et al., J Dairy Sci 1997, 80, 2270-2281.
[38] Morgan, F., Leonil, J., Molle, D., Bouhallab, S., Biochem Biophys Res Commun 1997, 236, 413-417.
[39] Marvin, L. F., Parisod, V., Fay, L. B., Guy, P. A., Electrophoresis 2002, 23, 2505-2512.

1. A method of resolving a polyhydric species present in a sample by gel electrophoresis, the method comprising:
(a) loading an electrophoresis gel with the sample containing the polyhydric species, wherein the electrophoresis gel is formed from a copolymer of a boronic acid species and a polymerisable linker, the boronic acid species being present in the copolymer at between 0.1% and 1.9% dry weight;
(b) applying an electric field across the gel to cause the polyhydric species to migrate across the gel; wherein the boronic acid species reversibly interacts with the hydroxyl groups present in the polyhydric species to cause different polyhydric species migrate through the gel at different speeds.

2. The method of claim 1, wherein the reaction to form the electrophoresis gel includes a template molecule that becomes incorporated into the electrophoresis gel, thereby providing cavities in the gel that are adapted to reversibly interact with one or more of the polyhydric species present in the sample having structures which are similar to the template molecule.

3. The method of claim 2, wherein the template molecule is a polyhydric species that forms boronic esters or boronic ester analogues with the boronic acid species.

4. The method according to claim 1, wherein the boronic acid species interacts with the hydroxyl groups present in the polyhydric species to reversibly form boronate esters or boronate ester analogues.

5. The method according to claim 1, wherein the polyhydric species comprises two hydroxyl groups in a 1, 1 or 1, 2 or 1, 3 or 1,4 positional relationship with each other.

6. The method according to claim 5, wherein said two hydroxyl groups are cis to each other.

7. The method according to claim 1, wherein the polyhydric species is a carbohydrate containing species or a phosphate containing species.

8. The method according to claim 7, wherein the carbohydrate containing species is a saccharide, a glycoprotein, DNA or RNA.

9. The method according to claim 1 wherein the polyhydric species is the product of post-translational modification of a polypeptide.

10. The method according to claim 9, wherein the polyhydric species is a glycosylated polypeptide, a gluconoylated polypeptide and/or a phosphorylated polypeptide.

11. The method according to claim 1, wherein the sample contains a plurality of different polyhydric species, and the method comprises separating the different polyhydric species according to their different migration speeds through the gel.

12. The method according to claim 11, wherein different polyhydric species migrate through the gel at different speeds according to their mass/charge ratio and/or their boron affinity.

13. The method according to claim 2, wherein different species applied to the gels migrate at different speeds according to whether they include a polyhydric species in the sample having a structure which is similar to the template molecule.

14. The method according to claim 1 which comprises one or more of the initial steps of:
(i) mixing the copolymer of the boronic acid species and the polymerisable linker, optionally in the presence of the template molecule, with a solvent for casting the gel; and/or
(ii) dissolving the copolymer in the solvent; and/or
(iii) casting the solution to produce the gel; and/or
(iv) optionally washing to remove the template molecule.

15. The method according to claim 1 which comprises the initial step of forming the copolymer from the boronic acid species, the polymerisable linker and optionally a polymerisable cross-linker, which polymerisable cross-linker may be a bisacrylamide linker such as methylene bisacrylamide.

16. The method according to claim 1, wherein the method comprises labelling the polyhydric species.

17. The method according to claim 16, wherein the label is any fluorescent or visible label.

18. The method according to claim 16, wherein the label is a neutral label.

19. The method according to claim 17, wherein the fluorescent label is 2-aminoacridone.

20. The method according to claim 1 further comprising detecting one or more of the polyhydric species resolved or separated on the gel.

21. The method according to claim 1, further comprising correlating the presence or amount of one or more of the polyhydric species as a marker of a disease, condition or biological process.

22. The method according to claim 19, wherein the disease, condition or biological process is selected from cancer, microbial infection, Alzheimer's disease, diabetes, cardiovascular disease and ageing, including diabetes-related ageing.

23. A method for diagnosing a patient suspected of having a disease associated with a polyhydric species, the method comprising:
(a) loading an electrophoresis gel with a sample containing the polyhydric species obtained from the patient, wherein the electrophoresis gel is formed from a copolymer of boronic acid species and an polymerisable linker, the boronic acid species being present in the copolymer at between 0.1% and 1.9% dry weight;
(b) applying an electric field across the gel to cause the polyhydric species to migrate through the gel, wherein the boronic acid species reversibly interacts with hydroxyl groups present in the polyhydric species to cause different polyhydric species to migrate through the gel at different speeds, thereby allowing the polyhydric species to be resolved;
(c) detecting the polyhydric species resolved on the gel;
(d) correlating the presence or amount of one or more of the polyhydric species as a marker of a disease or condition.

24. The method of claim 23, wherein the reaction to form the electrophoresis gel includes a template molecule that becomes incorporated into the electrophoresis gel, thereby providing cavities in the gel that are adapted to reversibly interact with one or more of the polyhydric species present in the sample having structures which are similar to the template molecule.

25. The method of claim 24, wherein the template molecule is a polyhydric species that forms boronic esters or boronic ester analogues with the boronic acid species.

26. The method according to claim 23, wherein the disease or condition is cancer or a microbial infection.

27. A method of making a gel for resolving a polyhydric species present in a sample by gel electrophoresis, the method comprising:
(i) mixing a copolymer of boronic acid species and a polymerisable linker with a solvent for casting the gel wherein the boronic acid species is present in the copolymer at between 0.1% and 1.9% dry weight; and/or
(ii) dissolving the copolymer in the solvent; and/or (i[upsilon]) casting the solution to produce the gel; wherein the boronic acid species is capable of reversibly interacting with hydroxyl groups present in the polyhydric species to cause different polyhydric species in the sample to migrate through the gel at different speeds.

28. The method of claim 27, wherein the reaction to form the electrophoresis gel includes a template molecule that becomes incorporated into the electrophoresis gel, thereby providing cavities in the gel that are adapted to reversibly interact with one or more of the polyhydric species present in the sample having structures which are similar to the template molecule.

29. The method of claim 28, wherein the template molecule is a polyhydric species that forms boronic esters or boronic ester analogues with the boronic acid species.

30. The method according to claim 27 which comprises the initial step of forming the copolymer from the boronic acid species, the polymerisable linker and optionally a polymerisable cross-linker, which polymerisable cross-linker may be a bisacrylamide linker such as methylene bisacrylamide.

31. The method according to claim 1, wherein the boronic acid species is capable of polymerisation with an acrylamide monomer to form the electrophoresis gel.

32. The method according to claim 1 wherein the boronic acid species comprises substituted or unsubstituted aryl boronic acid, or a boronate ester thereof.

33. The method according to claim 27, wherein the boronic acid species comprises substituted or unsubstituted phenyl boronic acid, or a boronate ester thereof.

34. The method according to claim 27, wherein the boronic acid species is a boronic acid acrylamide, or a boronate ester thereof.

35. The method according to claim 34 wherein the boronic acid acrylamide is ortho-, meta-, or para-methacrylamido phenylboronic acid, or a boronate ester thereof.

36. The method according to claim 1 wherein the boronic acid species is present in the copolymer at between 0.1% and 1.5% dry weight.

37. The method according to claim 36, wherein the boronic acid group is used at between 0.5% and 1.0% dry weight.

38. The method according to claim 1, wherein the copolymer is a copolymer of the boronic acid species, the polymerisable linker and a polymerisable cross-linker.

39. The method according to claim 38, wherein the polymerisable cross-linker is a bisacrylamide monomer, such as methylene bisacrylamide.

40. The method according to claim 1, wherein the polymerisable linker is an acrylamide monomer.

41. An electrophoresis gel for resolving polyhydric species, the electrophoresis gel being obtainable by copolymerising a boronic acid species capable of polymerisation with a polymerisable linker, wherein the boronic acid species is present at between 0.1% and 1.9% dry weight.

42. The electrophoresis gel of claim 41, wherein the electrophoresis gel is obtainable by copolymerising the boronic acid species and the polymerisable linker in the presence of a template molecule, so that the template molecule incorporates into the electrophoresis gel, thereby providing cavities in the gel that are adapted to reversibly interact with one or more of the polyhydric species present in a sample having structures which are similar to the template molecule.

43. The electrophoresis gel according to claim 42, wherein the template molecule is a polyhydric species that forms boronic esters or boronic ester analogues with the boronic acid species.

44. The electrophoresis gel according to claim 41, wherein the polymerisable linker is an acrylamide monomer.

45-75. (canceled)
